SEQUENCE LISTING (27 sequences)

SEQ ID NO: 1 - protein, 13 aa, Artificial Sequence
  indolicidin
ILPWKWPWWPWRR

SEQ ID NO: 2 - protein, 12 aa, Artificial Sequence
  JK1
VFLRRIRVIVIR

SEQ ID NO: 3 - protein, 12 aa, Artificial Sequence
  JK2
VFWRRIRVWVIR

SEQ ID NO: 4 - protein, 12 aa, Artificial Sequence
  JK3
VQLRAIRVRVIR

SEQ ID NO: 5 - protein, 12 aa, Artificial Sequence
  JK4
VQLRRIRVWVIR

SEQ ID NO: 6 - protein, 12 aa, Artificial Sequence
  JK5
VQWRAIRVRVIR

SEQ ID NO: 7 - protein, 12 aa, Artificial Sequence
  JK6
VQWRRIRVWVIR

SEQ ID NO: 8 - DNA, 20 bp, Artificial Sequence
  CpG 1826
tccatgacgttcctgacgtt

SEQ ID NO: 9 - DNA, 22 bp, Artificial Sequence
  CpG 2007
tcgtcgttgtcgttttgtcgtt

SEQ ID NO: 10 - DNA, 24 bp, Artificial Sequence
  CpG 7909 or 10103
tcgtcgttttgtcgttttgtcgtt

SEQ ID NO: 11 - DNA, 21 bp, Artificial Sequence
  CpG 8954
ggggacgacgtcgtggggggg

SEQ ID NO: 12 - DNA, 22 bp, Artificial Sequence
  CpG 2395 or 10101
tcgtcgttttcggcgcgcgccg

SEQ ID NO: 13 - DNA, 32 bp, Artificial Sequence
  Non-CpG oligonucleotide
aaaaaaggtacctaaatagtatgtttctgaaa

SEQ ID NO: 14 - protein, 27 aa, Artificial Sequence
  BMAP27
GRFKRFRKKFKKLFKKLSPVIPLLHLG

SEQ ID NO: 15 - protein, 28 aa, Artificial Sequence
  BMAP28
GGLRSLGRKILRAWKKYGPIIVPIIRIG

SEQ ID NO: 16 - protein, 12 aa, Artificial Sequence
  Bactenicin 2a (Bac2a)
RLARIVVIRVAR

SEQ ID NO: 17 - protein, 37 aa, Artificial Sequence
  human LL-37 peptide
LLGDFFRKSKEKIGKEFKRIVQRIKDFLRNLVPRTES

SEQ ID NO: 18 - protein, 12 aa, Artificial Sequence
  HH2 peptide
VQLRIRVAVIRA

SEQ ID NO: 19 - protein, 12 aa, Artificial Sequence
  IDR-1002
VQRWLIVWRIRK

SEQ ID NO: 20 - protein, 12 aa, Artificial Sequence
  1018 peptide
VRLIVAVRIWRR

SEQ ID NO: 21 - protein, 7 aa, Artificial Sequence
  HH18 peptide
IWVIWRR

SEQ ID NO: 22 - protein, 34 aa, Artificial Sequence
  Nisin Z
  misc_feature (2)..(2): Xaa is Dhb
  misc_feature (5)..(5): Xaa is Dha
  misc_feature (8)..(8): Xaa is Abu
  misc_feature (13)..(13): Xaa is Abu
  misc_feature (23)..(23): Xaa is Abu
  misc_feature (25)..(25): Xaa is Abu
  misc_feature (33)..(33): Xaa is Dha
IXAIXLAXPGAKXGALMGANMKXAXANASINVXK

SEQ ID NO: 23 - protein, 12 aa, Artificial Sequence
  conserved motif
  misc_feature (2)..(3): Xaa can be any amino acid
  misc_feature (5)..(5): Xaa can be any amino acid
  misc_feature (9)..(9): Xaa can be any amino acid
VXXRXIRVXVIR

SEQ ID NO: 24 - protein, 13 aa, Artificial Sequence
  HH111 peptide
ILKWKWPWWPWRR

SEQ ID NO: 25 - protein, 13 aa, Artificial Sequence
  HH113 peptide
ILPWKKPWWPWRR

SEQ ID NO: 26 - protein, 13 aa, Artificial Sequence
  HH970 peptide
ILKWKWPWWKWRR

SEQ ID NO: 27 - protein, 13 aa, Artificial Sequence
  HH1010 peptide
ILRWKWRWWRWRR

(19) United States
(12) Patent Application Publication
(10) Pub. No.: US 2021/0267890 A1
(43) Pub. Date: Sep. 2, 2021

(54) MUCOADHESIVE LIPIDIC DELIVERY SYSTEM

(71) Applicant: University of Saskatchewan, Saskatoon (CA)

(72) Inventors: Ellen K. Wasan, Saskatoon (CA); Kishor M. Wasan, Saskatoon (CA); Volker Gerdts, Saskatoon (CA); Stacy Strom, Saskatoon (CA)

(21) Appl. No.: 17/276,649

(22) PCT Filed: Sep. 20, 2019
(86) PCT No.: PCT/CA2019/051347

§ 371 Date: Mar. 16, 2021

Related U.S. Application Data
(60) Provisional application No. 62/733,881, filed on Sep. 20, 2018.

(51) Int. Cl. A61K 9/127; A61K 39/39; A61K 9/00

(52) U.S. Cl. CPC A61K 9/127; A61K 39/39; A61K 9/0043; A61K 9/0019; A61K 9/006

(57) ABSTRACT

Methods and compositions for enhancing an immune response, such as a mucosal immune response, to a selected antigen are described. The methods are useful for the treatment and prevention of microbial infections, such as infections caused by bacteria, viruses, fungi and parasites.

### TECHNICAL FIELD

The present invention pertains generally to compositions for enhancing immune responses to antigens. In particular, the invention relates to combination adjuvant compositions delivered using mucoadhesive cationic lipidic carriers, for use as vaccine adjuvants to stimulate mucosal immunity.

### BACKGROUND

Killed or subunit vaccines are often poorly immunogenic, and can result in weak and transient T-cell responses, thus requiring adjuvants to boost the immune response. Adjuvants are therefore crucial components of many vaccines. They are used to improve the immunogenicity of vaccines with the aim of conferring long-term protection, enhancing the efficacy of vaccines in newborns, elderly, or immunocompromised persons, and reducing the amount of antigen or the number of doses required to elicit effective immunity.

However, many currently available vaccines include adjuvants that are suboptimal with respect to the quality and magnitude of immune responses they induce. For example, alum, one of the few approved adjuvants for use in humans in the United States, induces good Th2 type immune responses but is not a potent adjuvant for Th1-type immune responses (HogenEsch et al., Vaccine (2002) 20 Suppl 3:S34-39). Thus, there is a need for additional effective and safer adjuvants.

It is now widely recognized that especially for respiratory diseases, the induction of both local and systemic immunity can substantially improve the level of protection. The advantage of mucosal administration, such as intranasal delivery, lies in the ability to induce both local and systemic immunity, while intramuscular immunization only induces systemic immunity. Indeed, more and more vaccines are now administered mucosally, in both humans and animals. For intranasal vaccines to be effective, it is necessary that the vaccine be delivered in a carrier that is adherent to the nasal mucous and can penetrate to the mucosa itself, and furthermore that the immunostimulatory effects of the adjuvant be maximized.

Recently, a combination adjuvant platform has been developed that includes three components: (1) an immunostimulatory molecule, such as a CpG or poly(I:C) (polyinosinic-polycytidylic acid); (2) a polyphosphazene such as poly[di(sodium carboxylatophenoxy)phosphazene] (PCPP) or a poly(di-4-oxyphenylproprionate)phosphazene (PCEP) (as a sodium salt or in the acidic form); and (3) antimicrobial molecules capable of killing a broad spectrum of microbes known as “host defense peptides.” See, e.g., U.S. Pat. Nos. 9,408,908 and 9,061,001, incorporated herein by reference in their entireties. This triple adjuvant forms a stable complex and has been demonstrated to be highly effective in a wide range of human and animal vaccines following intramuscular or subcutaneous administration. See, e.g., Garg et al., J. Gen. Virol. (2014) 95:301-306. This triple adjuvant composition, when used with various vaccine antigens, induces effective long-term humoral and cellular immunity. Moreover, the adjuvant platform is suitable for maternal immunization and is highly effective in neonates even in the presence of maternal antibodies. However, the efficacy by the nasal route to maximize mucosal immunity still requires enhancement.

Despite the various advances in adjuvant technology, there remains a need for safe and effective methods to prevent infectious diseases. Thus, the wide-spread availability of new adjuvant delivery methods for mucosal immunity is highly desirable.

### SUMMARY OF THE INVENTION

The present invention is based in part, on the discovery that the use of a combination adjuvant, including a host defense peptide, a polyanionic polymer such as a polyphosphazene, a nucleic acid sequence possessing immunostimulatory properties (ISS), such as poly(I:C), formulated with a mucoadhesive cationic lipidic carrier, provides for significantly higher antibody titers to a coadministered antigen when delivered intramuscularly or mucosally, as compared to those observed without such components. The adjuvant composition provides a safe and effective approach for enhancing the immunogenicity of a variety of vaccine antigens for use in both prophylactic and therapeutic compositions.

Accordingly, in one embodiment, a mucoadhesive lipidic carrier system is provided. The mucoadhesive lipidic carrier system comprises a triple adjuvant composition that includes a host defense peptide, an immunostimulatory sequence and a polyanionic polymer, formulated with a mucoadhesive lipidic carrier. The mucoadhesive lipidic carrier system is capable of enhancing an immune response to a selected antigen. In certain embodiments, the mucoadhesive lipidic carrier system is capable of enhancing the immune response when administered mucosally. In some embodiments, the mucoadhesive lipidic carrier system is capable of enhancing the immune response when administered intramuscularly. In certain embodiments, the mucoadhesive lipidic carrier of the system comprises a cationic liposome, such as, but not limited to, a mucoadhesive cationic lipid carrier comprising one or more cationic lipids selected from 1,2-dioleoyl-3-trimethylammonium-propane (DOTAP); 3β-[N—(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); or ceramide carbamoyl-spermine (CCS).

In additional embodiments, the mucoadhesive lipidic carrier of the system is comprised of DDAB and DOPE; DDAB, EPC and DOPE; SA and Chol; EPC and Chol; or SA/EPC and Chol.

In yet further embodiments, the host defense peptide of the mucoadhesive lipidic carrier system is IDR-1002 (SEQ ID NO:19).

In additional embodiments, the immunostimulatory sequence of the mucoadhesive lipidic carrier system is polyinosinic-polycytidylic acid (poly(I:C)) or CpG.

In further embodiments, the polyphosphazene of the mucoadhesive lipidic carrier system is poly(di-4-oxyphenylproprionate)phosphazene (PCEP), such as a sodium salt of PCEP.

In additional embodiments, the mucoadhesive lipidic carrier system comprises an antigen from a pathogen that invades mucosal tissue, such as an antigen is from a virus, bacteria, parasite or fungus.

In yet additional embodiments, a mucoadhesive cationic liposome carrier system is provided. The cationic liposome carrier system comprises (a) DDAB and DOPE; DDAB, EPC and DOPE; SA and Chol; EPC and Chol; or SA/EPC and Chol; (b) IDR-1002 (SEQ ID NO:19); (c) poly(I:C); (d) PCEP (such as a sodium salt of PCEP); and (e) an antigen from a pathogen that invades mucosal tissue. In certain embodiments, the antigen is from a virus, bacteria, parasite or fungus.

In further embodiments, a composition is provided that comprises mucoadhesive lipidic carrier systems as described herein; and a pharmaceutically acceptable excipient. In certain embodiments, the average diameter of the mucoadhesive lipidic carrier systems in the composition is less than 200 nanometers.

In additional embodiments, a method of enhancing an immune response to a selected antigen is provided. The method comprises administering to a subject the composition; and a selected antigen. In certain embodiments, the administering is done mucosally. In certain embodiments, the administering is done intranasally. In certain embodiments, the administering is done intramuscularly.

These and other embodiments of the subject invention will readily occur to those of skill in the art in view of the disclosure herein.

### BRIEF DESCRIPTION OF THE FIGURES

FIG. 1 shows the stability of the lipidic triple adjuvant particles (L-TriAdj) over 24 hours, assessed by zeta potential. Data represent mean+/−SD (n=3).

FIG. 2 shows the results of mucin binding studies using DDAB/DOPE 50:50 and various compositions, as described in the examples.

FIG. 3 shows the results of mucin binding studies using DDAB/DOPE 75:25 and various compositions as described, in the examples.

FIG. 4 shows the results of mucin binding studies using DDAB/EggPC/DOPE 40:50:10 and various compositions, as described in the examples.

FIG. 5 shows the results of mucin binding studies using EggPC/Chol 90:10 and various compositions, as described in the examples.

FIG. 6 shows the results of an MTS cytotoxicity assay where TriAdj content was constant at 0.5:1:0.5 (μg:μg:μg)/well, as described in the examples.

FIG. 7 shows the results of an MTS cytotoxicity assay where TriAdj content was constant at 0.25:0.5:0.25 μg:μg:μg/well, as described in the examples.

FIGS. 8A-8J show the immunological responses obtained in animals following intranasal administration as described in the examples. FIGS. 8A and 8F show the ELISA results of IgG2a (8A) and IgG1 (8F) response in mice after nasal vaccine administration of TriAdj with ovalbumin (Ova) as the antigen and either 1:2:1 or 5:10:5 μg weight ratio TriAdj per dose. L-TriAdj was formulated with DDAB/DOPE (50:50 mol/mol) or DDAB/EPC/DOPE (40/50/10 mol/mol/mol). For all other figures (8B, 8C, 8D, 8E, 8G, 8H, 8I, 8J) the dose of TriAdj was 5:10:5 μg except 8B and 8G (PBS control immunization). Data in FIGS. 8B, 8C, 8D, 8E, 8G, 8H, 8I and 8J represent ELISpot results from spleen lymphocytes harvested from the vaccinated mice, showing Ova antigen-stimulated secretion of IFN-γ (left side of the figure) or IL-5 (right side), respectively. Data represent response from triplicate samples from individual mice and the horizontal bar represents the median value (n=8). ● Saline control; ▪ Antigen only; ▴ TriAdj; ▾ L-TriAdj DDAB/DOPE (2 μg peptide); ♦ L-TriAdj DDAB/DOPE (10 μg peptide); [symbol] L-TriAdj DDAB/EPC/DOPE (2 μg peptide); * L-TriAdj DDAB/EPC/DOPE (10 μg peptide).

FIG. 9 shows the effect of TriAdj dose on the immune response to the adjuvanted ovalbumin vaccine in mice. Data represent the fourth quartile of IFN-γ response from each treatment group (n=8/group).

FIG. 10 shows the ratio of ELISpot values for interferon-γ (INF) and interleukin-5 (IL-5) for each mouse vaccinated with the triple adjuvant composition (TriAdj) or lipidic triple adjuvant particles that included ovalbumin antigen (Ova) (L-TriAdj+Ova), as described in the examples. Results are expressed as mean±SD (n=7). TriAdj dose of 1:2:1 or 5:10:5 μg and lipid composition are as in FIG. 8. The spleen lymphocytes from the vaccinated mice were exposed in triplicate to 5 or 10 μg ovalbumin ex vivo and secretion of IL5 and IFN were measured. The ratio of these values reflects the balance of cellular (Th1) versus humoral (Th2) type response. *Significantly different from L-TriAdj DDAB/DOPE with 5:10:5 μg TriAdj and stimulated with 5 μg Ova (p=0.05). Peptide dose of 2 or 10 μg within the TriAdj and lipid composition are as in FIG. 8.

FIGS. 11A-11J show the immunological responses obtained in animals following intranasal administration as described in the examples. FIGS. 11A and 11B show the ELISA results of IgG2a (11A) and IgG1 (11B) response in mice after intranasal vaccine administration of TriAdj with ovalbumin (Ova) as the antigen and either 1 μg or 10 μg Ova/dose and TriAdj comprised of 5 μg poly(I:C):10 μg IDR-1002:5 μg PCEP sodium salt per dose. L-TriAdj was formulated with DDAB/DOPE (50:50 mol/mol), at 0, 4 and 10 weeks. Data in 11C, 11D, 11E, 11F, 11G, 11H, 11I and 11J represent ELISpot results from spleen lymphocytes harvested from the vaccinated mice, showing ex vivo Ova antigen-stimulated secretion of IFN-γ (left side of the figure) or IL5 (right side), respectively. Data represent response from triplicate samples from individual mice and the horizontal bar represents the median value (n=8). ●: Ova 1 μg only; ▪: Ova 10 μg only; ▴: Ova 1 μg+TriAdj MP; ▾: Ova 10 μg+TriAdj MP; ♦: Ova 1 g+L-TriAdj; [symbol] : Ova 10 μg+L-TriAdj; black star: Ova 1 μg+TriAdj; ∘: Ova 10 μg+TriAdj.

FIG. 12 shows that adjuvant activity at 4 weeks post-vaccination is greater in mice receiving Ova+L-TriAdj vaccine, based on IgG2a serum levels. Data represent log values (n=8); X represents median value.

FIG. 13 shows the ELISA results of serum IgA response in mice administered either 1 μg (FIG. 13A) or 10 (FIG. 13B) μg Ova/dose, and TriAdj comprised of 5 μg poly(I:C):10 μg IDR-1002:5 μg PCEP sodium salt per dose, either formulated with DDAB/DOPE (50:50 mol/mol, labelled as L-TriAdj), as microparticles (labelled as TriAdj MP) or in solution (labelled as TriAdj).

FIG. 14 shows the ELISA results of serum IgG1 response in mice as described in the examples after intranasal or intramuscular vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP sodium salt) or TriAdj microparticles (5 μg poly(I:C):10 μg IDR-1002:5 μg PCEP sodium salt), before immunization (FIG. 14A), at 4 weeks (FIG. 14B), 6 weeks (FIG. 14C) and 10 weeks (FIG. 14D). ●: Ova 10 μg+L-TriAdj, delivered intranasally; ▪: Ova 10 μg+L-TriAdj, delivered intramuscularly; ▴: Ova 10 μg+TriAdj MP (5:10:5) delivered intranasally; ▾: Ova 10 μg+TriAdj MP (5:10:5) delivered intramuscularly; +: Ova 10 μg delivered intramuscularly. Data represent response from samples from individual mice and the horizontal bar represents the median value (n=8).

FIG. 15 shows the ELISA results of serum IgG2a response in mice as described in the examples after intranasal or intramuscular vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP), before immunization (FIG. 15A), at 4 weeks (FIG. 15B), 6 weeks (FIG. 15C) and 10 weeks (FIG. 15D). ●: Ova 10 μg+L-TriAdj, delivered intranasally; ▪: Ova 10 μg+L-TriAdj, delivered intramuscularly; ▴: Ova 10 μg+TriAdj MP (5:10:5) delivered intranasally; ▾: Ova 10 μg+TriAdj MP (5:10:5) delivered intramuscularly; +: Ova 10 μg delivered intramuscularly. Data represent response from samples from individual mice and the horizontal bar represents the median value (n=8).

FIG. 16 shows the ELISA results of serum IgA response in mice as described in the examples after intranasal or intramuscular vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP), before immunization (FIG. 16A), at 4 weeks (FIG. 16B), 6 weeks (FIG. 16C) and 10 weeks (FIG. 16D). ●: Ova 10 μg+L-TriAdj, delivered intranasally; ▪: Ova 10 μg+L-TriAdj, delivered intramuscularly; ▴: Ova 10 μg+TriAdj MP (5:10:5) delivered intranasally; ▾: Ova 10 μg+TriAdj MP (5:10:5) delivered intramuscularly; +: Ova 10 μg delivered intramuscularly. Data represent response from samples from individual mice and the horizontal bar represents the median value (n=8).

FIG. 17 shows the ELISA results of IgG1, IgG2a and IgA response in intranasal (IN) washes of mice after intranasal or intramuscular vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP) 10 weeks after the first immunization. IN wash IgG1 response is presented in FIG. 17A, IN wash IgG2a response in FIG. 17B and IN wash IgA response in FIG. 17C. ●: Ova 10 μg+L-TriAdj, delivered intranasally; ▪: Ova 10 μg+L-TriAdj, delivered intramuscularly; ▴: Ova 10 μg+TriAdj MP (5:10:5) delivered intranasally; ▾: Ova 10 μg+TriAdj MP (5:10:5) delivered intramuscularly; +: Ova 10 μg delivered intramuscularly. Data represent response from samples from individual mice and the horizontal bar represents the median value (n=8).

FIG. 18 shows the ELISA results of IgG1, IgG2a and IgA response in bronchio-alveaolar lavages (BAL)s of mice after intranasal or intramuscular vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP) 10 weeks after the first immunization. BAL IgG1 response is presented in FIG. 18A, BAL IgG2a response in FIG. 18B and BAL IgA response in FIG. 18C. Data represent response from samples from individual mice and the horizontal bar represents the median value (n=8).

FIG. 19 represents ELISpot results from spleen lymphocytes harvested from the vaccinated mice at 10 weeks, showing ex vivo Ova antigen-stimulated secretion of IFN-γ. Mice had been vaccinated by intranasal (IN) or intramuscular (IM) vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP). ELISpot stimulation agents are: ●: media (negative control); ▪: Ova 5 μg/mL; ▴: Ova 10 μg/mL. Data represent response from triplicate samples from individual mice and the horizontal bar represents the median value (n=8).

FIG. 20 represents ELISpot results from spleen lymphocytes harvested from the vaccinated mice at 10 weeks, showing ex vivo Ova antigen-stimulated secretion of IL5. Mice had been vaccinated by intranasal (IN) or intramuscular (IM) vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP). ELISpot stimulation agents are: ●: media (negative control); ▪: Ova 5 μg/mL; ▴: Ova 10 μg/mL. Data represent response from triplicate samples from individual mice and the horizontal bar represents the median value (n=8).

FIG. 21 represents ratios of IFNγ and IL5 ELISpot results after ex vivo Ova antigen-stimulated secretion from spleen lymphocytes harvested from the vaccinated mice at 10 weeks. Mice had been vaccinated by intranasal (IN) or intramuscular (IM) vaccine administration of 10 μg ovalbumin (Ova) as the antigen and either L-TriAdj (formulated with DDAB/DOPE (50:50 mol/mol) and TriAdj as 5 μg poly(I:C):10 μg IDR-1002:5 μg PCEP) or TriAdj microparticles (5 μg poly(I:C):10 μg IDR-1002:5 μg PCEP). ELISpot stimulation agents are: ●: media (negative control); ▪: Ova 5 μg/mL; ▴: Ova 10 μg/mL. Data represent response from triplicate samples from individual mice and the horizontal bar represents the median value (n=8).

FIG. 22 shows representative polyphosphazene compounds for use in the present formulations.

### DETAILED DESCRIPTION OF THE INVENTION

The practice of the present invention will employ, unless otherwise indicated, conventional methods of microbiology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, current Edition); A. L. Lehninger, Biochemistry (Worth Publishers, Inc., current addition); Sambrook, et al., Molecular Cloning: A Laboratory Manual (current addition); Methods In Enzymology (S. Colowick and N. Kaplan eds., Academic Press, Inc.).

All publications, patents and patent applications cited herein, whether supra or infra, are hereby incorporated by reference in their entireties.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| *Tyrosine: Tyr (Y) | Valine: Val (V) |
| Dehydroalanine (Dha) | Dehydrobutyrine (Dhb) |

TABLE 1
Sequences presented herein:

| SEQ ID NO | SEQUENCE | NAME |
|---|---|---|
| 1 | ILPWKWPWWPWRR | indolicidin |
| 2 | VFLRRIRVIVIR | JK1 |
| 3 | VFWRRIRVWVIR | JK2 |
| 4 | VQLRAIRVRVIR | JK3 |
| 5 | VQLRRIRVWVIR | JK4 |
| 6 | VQWRAIRVRVIR | JK5 |
| 7 | VQWRRIRVWVIR | JK6 |
| 8 | TCCATGACGTTCCTGACGTT | CpG 1826 |
| 9 | TCGTCGTTGTCGTTTTGTCGTT | CpG 2007 |
| 10 | TCGTCGTTTTGTCGTTTTGTCGTT | CpG 7909 or 10103 |
| 11 | GGGGACGACGTCGTGGGGGGG | CpG 8954 |
| 12 | TCGTCGTTTTCGGCGCGCGCCG | CpG 2395 or 10101 |
| 13 | AAAAAAGGTACCTAAATAGTATGTTTCTGAAA | Non-CpG oligo |
| 14 | GRFKRFRKKFKKLFKKLSPVIPLLHLG | BMAP27 |
| 15 | GGLRSLGRKILRAWKKYGPIIVPIIRIG | BMAP28 |
| 16 | RLARIVVIRVAR | Bactenicin 2a (Bac2a) |
| 17 | LLGDFFRKSKEKIGKEFKRIVQRIKDFLRNLVPRTES | human LL-37 |
| 18 | VQLRIRVAVIRA | HH2 |
| 19 | VQRWLIVWRIRK | 1002 |
| 20 | VRLIVAVRIWRR | 1018 |
| 21 | IWVIWRR | HH18 |
| 22 | Ile-Dhb-Ala-Ile-Dha-Leu-Ala-Abu-Pro-Gly-Ala-Lys-Abu- | Nisin Z |
| | Gly-Ala-Leu-Met-Gly-Ala-Asn-Met-Lys-Abu-Ala-Abu-Ala- | |
| | Asn-Ala-Ser-Ile-Asn-Val-Dha-Lys | |
| 23 | V**R*IRV*VIR, * = any amino acid | conserved motif |
| 24 | ILKWKWPWWPWRR | HH111 |
| 25 | ILPWKKPWWPWRR | HH113 |
| 26 | ILKWKWPWWKWRR | HH970 |
| 27 | ILRWKWRWWRWRR | HH1010 |

### I. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a host defense peptide” includes a mixture of two or more host defense peptides, and the like.

By “host defense peptide” or “HDP” is meant any of the various host defense peptides that have the ability to enhance an immune response to a co-administered antigen. The DNA and corresponding amino acid sequences for various host defense peptides are known and described in detail below. Host defense peptides for use in the present methods include the full-length (i.e., a prepro sequence if present, the entire prepro molecule) or substantially full-length proteins, as well as biologically active fragments, fusions or mutants of the proteins. The term also includes postexpression modifications of the polypeptide, for example, glycosylation, acetylation, phosphorylation and the like. Furthermore, for purposes of the present invention, a “host defense peptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification. It is readily apparent that the host defense peptides may therefore comprise an entire leader sequence, the mature sequence, fragments, truncated and partial sequences, as well as analogs, muteins and precursor forms of the molecule. The term also intends deletions, additions and substitutions to the reference sequence, so long as the molecule retains the desired biological activity.

By “poly(I:C) oligonucleotide” or “poly(I:C)” is meant a synthetic viral-like mis-matched double-stranded immunostimulatory ribonucleic acid containing strands of polyriboinosinic acid and polyribocytidylic acid that are held together by hydrogen bonds between purine and pyrimidine bases in the chains. Poly(I:C) has been found to have a strong interferon-inducing effect in vitro and is therefore of significant interest in infectious disease research.

By “CpG oligonucleotide”, “CpG”, or “CpG ODN” is meant an immunostimulatory nucleic acid containing at least one cytosine-guanine dinucleotide sequence (i.e., a 5′ cytidine followed by 3′ guanosine and linked by a phosphate bond) and which activates the immune system. An “unmethylated CpG oligonucleotide” is a nucleic acid molecule which contains an unmethylated cytosine-guanine dinucleotide sequence (i.e., an unmethylated 5′ cytidine followed by 3′ guanosine and linked by a phosphate bond) and which activates the immune system. A “methylated CpG oligonucleotide” is a nucleic acid which contains a methylated cytosine-guanine dinucleotide sequence (i.e., a methylated 5′ cytidine followed by a 3′ guanosine and linked by a phosphate bond) and which activates the immune system. CpG oligonucleotides are well known in the art and described in, e.g., U.S. Pat. Nos. 6,194,388; 6,207,646; 6,214,806; 6,218,371; 6,239,116; and 6,339,068; PCT Publication No. WO 01/22990; PCT Publication No. WO 03/015711; US Publication No. 20030139364, which patents and publications are incorporated herein by reference in their entireties.

By “polyphosphazene” is meant a high-molecular weight, water-soluble polymer, containing a backbone of alternating phosphorous and nitrogen atoms and organic side groups attached at each phosphorus atom. See, e.g., Payne et al., Vaccine (1998) 16:92-98; Payne et al., Adv. Drug. Deliv. Rev. (1998) 31:185-196. A number of polyphosphazenes are known and described in more detail below.

By “mucus membrane” or “mucosa” is meant any of the moist surfaces lining the walls of various body cavities such as, but not limited to, the respiratory tract, i.e., lungs and nasal passages; the gastrointestinal (GI) tract, including the mouth, esophagus, stomach, small intestine, large intestine, rectum and anus; the vagina; and the cornea. Mucus membranes consist of a connective tissue layer, the lamina propria (located below an epithelial layer), the surface of which is made moist usually by the presence of a mucus layer. The epithelia may be either single layered such as found in the stomach, small and large intestines and bronchi, or multilayered/stratified, such as present in the esophagus, vagina and eye. The former contains goblet cells that secrete mucus directly onto the epithelial surfaces while the latter contains or is adjacent to tissues that include specialized glands, such as salivary glands, that secrete mucus onto the epithelial surface. Mucus is present either as a gel layer adherent to the mucosal surface or as a luminal soluble or suspended form. The major components of all mucus gels are mucin glycoproteins, lipids, inorganic salts and water. The mucosa is the surface where most pathogens invade.

By “mucoadhesion” is meant the process of associating a substance with a mucus membrane. The mechanism of mucoadhesion is generally divided into two steps: the contact stage and the consolidation stage. The first step is characterized by contact between a mucoadhesive substance, in this case a mucoadhesive lipidic carrier system that includes an encapsulated triple adjuvant composition, and the mucus membrane, with spreading and swelling of the formulation. This initiates deep contact with the mucus layer. In the consolidation step, the mucoadhesive materials are activated by the presence of moisture. Moisture allows the mucoadhesive molecules to break free and link up by weak van der Waals and hydrogen bonds.

By “mucoadhesive lipidic carrier” is meant a particulate carrier composed of lipids, typically cationic lipids, such as a cationic liposome, wherein the carrier has the ability to associate with the mucosa through mucoadhesion, to stimulate a local, and in some cases a systemic, immune response when a selected co-administered antigen is present.

The “mucosal immune system” commonly called “MALT,” is an adaptive immune system located near the mucosa. The dominant antibody isotype of the mucosal immune system is IgA. This class of antibody is found in some mammals in two isotypic forms, IgA1 and IgA2. The expression of IgA differs between blood and mucosal secretions, the two main compartments in which it is found. In the blood, IgA is mainly found as a monomer and the ratio of IgA1 to IgA2 is approximately 4:1. In mucosal secretions, IgA is almost exclusively produced as a dimer and the ratio of IgA1 to IgA2 is approximately 3:2. A number of common intestinal pathogens possess proteolytic enzymes that can digest IgA1, whereas IgA2 is much more resistant to digestion.

By “intramuscular” is meant a method of injection or delivery of a desired composition, such as a mucoadhesive lipidic carrier system or a cationic mucoadhesive liposome carrier system, into muscle tissue of a patient. For example, a composition may be injected into the deltoid muscle of a patients arm.

By “antigen” or “immunogen” is meant a molecule, which contains one or more epitopes (defined below) that will stimulate a host's immune system to make a cellular antigen-specific immune response when the antigen is presented, and/or a humoral antibody response. The terms denote both subunit antigens, i.e., proteins which are separate and discrete from a whole organism with which the antigen is associated in nature, as well as killed, attenuated or inactivated bacteria, viruses, parasites or other microbes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses a therapeutic or immunogenic protein, or antigenic determinant in vivo, such as in gene therapy and nucleic acid immunization applications, is also included in the definition of antigen herein. Further, for purposes of the present invention, antigens can be derived from any of several known viruses, bacteria, parasites and fungi, as well as any of the various tumor antigens.

The term “derived from” is used to identify the original source of a molecule (e.g., bovine or human) but is not meant to limit the method by which the molecule is made which can be, for example, by chemical synthesis or recombinant means.

The terms “analog” and “mutein” refer to biologically active derivatives of the reference molecule that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined below. The term “mutein” refers to peptides having one or more peptide mimics (“peptoids”), such as those described in International Publication No. WO 91/04282. Preferably, the analog or mutein has at least the same desired activity as the native molecule. Methods for making polypeptide analogs and muteins are known in the art and are described further below.

The terms also encompass purposeful mutations that are made to the reference molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-10 conservative or non-conservative amino acid substitutions, or even up to about 15-20 conservative or non-conservative amino acid substitutions, or any integer between 5-20, so long as the desired function of the molecule remains intact. One of skill in the art can readily determine regions of the molecule of interest that can tolerate change by reference to Hopp/Woods and Kyte-Doolittle plots, well known in the art.

By “fragment” is intended a molecule consisting of only a part of the intact full-length polypeptide sequence and structure. The fragment can include a C-terminal deletion, an N-terminal deletion, and/or an internal deletion of the native polypeptide. A fragment will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains the ability to elicit the desired biological response.

By “immunogenic fragment” is meant a fragment of a parent molecule which includes one or more epitopes and thus can modulate an immune response or can act as an adjuvant for a co-administered antigen and/or is capable of inducing an adaptive immune response. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 81:3998-4002; Geysen et al., (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

Immunogenic fragments, for purposes of the present invention, will usually be at least about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the protein in question.

The term “epitope” refers to the site on an antigen or hapten to which specific B cells and T cells respond. The term is also used interchangeably with “antigenic determinant” or “antigenic determinant site.” Antibodies that recognize the same epitope can be identified in a simple immunoassay showing the ability of one antibody to block the binding of another antibody to a target antigen.

An “immunological response” to a composition is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or γδ T cells, directed specifically to an antigen or antigens included in the composition or vaccine of interest. Preferably, the host will display a protective immunological response to the microorganism in question, e.g., the host will be protected from subsequent infection by the pathogen and such protection will be demonstrated by either a reduction or lack of symptoms normally displayed by an infected host or a quicker recovery time.

The term “immunogenic” molecule refers to a molecule which elicits an immunological response as described above. An “immunogenic” protein or polypeptide, as used herein, includes the full-length sequence of the protein in question, including the precursor and mature forms, analogs thereof, or immunogenic fragments thereof.

An adjuvant composition comprising a host defense peptide, a polyphosphazene and an immunostimulatory sequence “enhances” or “increases” the immune response, or displays “enhanced” or “increased” immunogenicity vis-a-vis a selected antigen when it possesses a greater capacity to elicit an immune response than the immune response elicited by an equivalent amount of the antigen when delivered without the adjuvant composition. Such enhanced immunogenicity can be determined by administering the antigen and adjuvant composition, and antigen controls to animals and comparing antibody titers against the two using standard assays such as radioimmunoassay and ELISAs, well known in the art.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, metal chelation chromatography, reversed phase chromatography, hydrophobic interaction chromatography, and sedimentation according to density.

By “isolated” is meant that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “effective amount” or “pharmaceutically effective amount” of a composition, or a component of the composition, refers to a nontoxic but sufficient amount of the composition or component to provide the desired response, such as enhanced immunogenicity, and, optionally, a corresponding therapeutic effect. The exact amount required will vary from subject to subject, depending on the species, age, and general condition of the subject, the severity of the condition being treated, and the particular components of interest, mode of administration, and the like. An appropriate “effective” amount in any individual case may be determined by one of ordinary skill in the art using routine experimentation.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

The term “treatment” as used herein refers to either (1) the prevention of infection or reinfection (prophylaxis), or (2) the reduction or elimination of symptoms of the disease of interest (therapy).

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that compositions including an immunostimulatory sequence, such as CpG or non-CpG oligonucleotides (e.g., poly(I:C)), a polyanionic polymer such as a polyphosphazene, and a host defense peptide, when administered using a mucoadhesive lipidic carrier, such as a cationic liposome, are useful for mucosal or intramuscular administration to enhance immune responses to a co-administered antigen. Thus, these systems can be used to confer protection against infections when delivered mucosally or intramuscularly, such as to membranes of the respiratory system, the GI tract, the urogenital tract, the eye, and the like.

The mucoadhesive lipidic carrier systems containing these triple adjuvant compositions are useful for the prevention and treatment of infectious diseases in humans and other animals, caused by a variety of pathogens that invade the mucosa, including diseases caused by bacteria, mycobacteria, viruses, fungi, parasites and the like, when used with a co-administered antigen.

The mucoadhesive lipidic carrier systems of the invention can be introduced into a subject using any of various mucosal or intramuscular delivery techniques, described more fully below. The systems can be used with one or multiple antigens or immunogens including polypeptide, polynucleotide, polysaccharide, or lipid antigens or immunogens, as well as with inactivated or attenuated pathogens, to produce an immune response, such as a mucosal immune response, in the subject to which the systems are delivered. The immune response can serve to protect against future infection or lessen or ameliorate the effects of infection.

In order to further an understanding of the invention, a more detailed discussion is provided below regarding host defense peptides, immunostimulatory sequences, polyanionic polymers, mucoadhesive lipidic carriers, and antigens for use in the subject compositions and methods.

Host Defense Peptides

As explained above, the methods and compositions of the present invention include host defense peptides. Over 400 of these anti-microbial proteins have been identified in plants, insects and animals. See, e.g., Boman, H. G., Annu. Rev. Immunol. (1995) 13:61-92; Boman, H. G., Scand. J. Immunol. (1998) 48:15-25; Broekaert et al., Plant. Physiol. (1995) 108:1353-1358; Steiner et al., Nature (1981) 292:246-248; Ganz et al., Curr. Opin. Immunol. (1994) 4:584-589; Lehrer et al., Curr. Opin. Immunol. (1999) 11:23-27. The two major families of mammalian host defense peptides are defensins and cathelcidins. See, e.g., Ganz et al., Curr. Opin. Immunol. (1994) 4:584-589; Lehrer et al., Curr. Opin. Immunol. (1999) 11:23-27; Ouellette et al., FASEB J. (1996) 10:1280-1289; Zanetti et al., FEBS Lett. (1995) 374:1-5.

Mammalian defensins are a family of cationic proteins that contain six highly conserved cysteine residues that form three pairs of intrachain-disulfide bonds. Mammalian defensins are classified into three subfamilies, α-, β-, and θ-defensins, based on the patterns of their intrachain-disulfide bridges, (Ganz et al., Curr. Opin. Immunol. (1994) 4:584-589; Lehrer et al., Curr. Opin. Immunol. (1999) 11:23-27; Tang et al., Science (1999) 286:498-502). The θ-defensin subfamily includes a cyclic molecule with its six cysteine residues linking C1 to C6, C2 to C5, and C3 to C4 (Tang et al., Science (1999) 286:498-502). The three disulfide bonds of α-defensins are paired C1 to C6, C2 to C4, and C3 to C5 (Ganz et al., Curr. Opin. Immunol. (1994) 4:584-589; Ouellette et al., FASEB J. (1996) 10:1280-1289; Zhang et al., Biochemistry (1992) 31:11348-11356). The disulfide bonds of β-defensins are C1 to C5, C2 to C4, and C3 to C6 (Ganz et al., Curr. Opin. Immunol. (1994) 4:584-589; Tang et al., J. Biol. Chem. (1993) 268:6649-6653).

More than 50 defensin family members have been identified in mammalian species. In humans, at least six α-defensins and three β-defensins have been identified (Ganz et al., Curr. Opin. Immunol. (1994) 4:584-589; Lehrer et al., Curr. Opin. Immunol. (1999) 11:23-27; Ouellette et al., FASEB J. (1996) 10:1280-1289; Ganz et al., J. Clin. Invest. (1985) 76:1427-1435; Wilde et al., J. Biol. Chem. (1989) 264:11200-11203; Mallow et al., J. Biol. Chem. (1996) 271:4038-4045; Bensch et al., FEBS Lett. (1995) 368:331-335; Larrick et al., Infect. Immun. (1995) 63:1291-1297). Non-limiting examples of human defensins include human α-defensins 1, 2, 3, and 4, also termed human neutrophil peptides (HNP)1, 2, 3, and 4; human α-defensins 5 and 6 (HD5 and 6); and human β-defensins (HBD) 1, 2 and 3.

Cathelicidins are a family of anti-microbial proteins with a putative N-terminal signal peptide, a highly conserved cathelin (cathepsin L inhibitor)-like domain in the middle, and a less-conserved, C-terminal, anti-microbial domain (Lehrer et al., Curr. Opin. Immunol. (1999) 11:23-27; Zanetti et al., FEBS Lett. (1995) 374:1-5). About 20 cathelicidin members have been identified in mammals, with at least one cathelicidin from humans (Zanetti et al., FEBS Lett. (1995) 374:1-5; Larrick et al., Infect. Immun. (1995) 63:1291-1297; Cowland et al., FEBS Lett. (1995) 368:173-176; Agerberth et al., Proc. Natl. Acad. Sci. USA (1995) 92:195-199). Cleavage of human cathelicidin (hCAP18) liberates its C-terminal, anti-microbial domain, a peptide called LL-37, with two N-terminal leucine residues. LL-37 is 37 amino-acid residues in length (Zanetti et al., FEBS Lett. (1995) 374:1-5; Gudmundsson et al., Eur. J. Biochem. (1996) 238:325-332).

Another group of host defense peptides contains a high percentage of specific amino acids, such as the proline-/arginine-rich bovine peptides, Bac2a, Bac5 and Bac7 (Gennaro et al., Infect. Immun. (1989) 57:3142-3146) and the porcine peptide PR-39 (Agerberth et al., Eur. J. Biochem. (1991) 202:849-854); and indolicidin which is a 13-amino acid host defense peptide with the sequence ILPWKWPWWPWRR (SEQ ID NO:1).

Other representative host defense peptides are presented in Table 1 and in the examples, such as peptide IDR-1002.

The host defense peptides for use herein can include a prepro sequence, a pro-protein without the pre sequence, or the mature protein without the prepro sequence. If a signal sequence is present the molecules can include, for example, the native signal sequence, along with a pro-sequence or the mature sequence. Alternatively, a host defense peptide for use herein can include a pro sequence or mature sequence with a heterologous signal sequence. Alternatively, host defense peptide for use herein can include only the sequence of the mature protein, so long as the molecule retains biological activity. Moreover, host defense peptides for use herein can be biologically active molecules that display substantial homology to the parent molecule, as defined above.

Thus, host defense peptides for use with the present invention can include, for example, the entire parent molecule, or biologically active fragments thereof, such as fragments including contiguous amino acid sequences comprising at least about 5-10 up to about 50 to the full-length of the molecule in question, or any integer there between. The molecule will typically include one or more epitopes. Such epitopes are readily identifiable using techniques well known in the art, such as using standard antigenicity and hydropathy plots, for example those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots. This program can be used with the following parameters: averaging results over a window of 7; determining surface probability according to Emini; chain flexibility according to Karplus-Schulz; antigenicity index according to Jameson-Wolf, secondary structure according to Garnier-Osguthorpe-Robson; secondary structure according to Chou-Fasman; and identifying predicted glycosylation sites. One of skill in the art can readily use the information obtained in combination with teachings of the present specification to identify antigenic regions which should be included in the molecules for use with the present invention.

Any of the above peptides, as well as fragments and analogs thereof, that display the appropriate biological activity, such as the ability to modulate an immune response, such as to enhance an immune response to a co-delivered antigen when delivered via a mucoadhesive lipidic carrier system that also contains the other components of the triple adjuvant as described herein, will find use in the present methods. Enhanced adjuvant activity displayed by delivery using a mucoadhesive lipidic carrier system can be elucidated by determining whether the composition of interest delivered with the carrier system and when co-delivered with the antigen of interest, possesses a greater capacity to elicit an immune response than the immune response elicited by an equivalent amount of the same composition delivered without a mucoadhesive lipidic carrier system. Such enhanced immunogenicity can be determined by comparing antibody titers or cellular immune response produced using standard assays such as radioimmunoassay, ELISAs, lymphoproliferation assays, and the like, well known in the art.

The host defense peptides for use with the present invention can be obtained using standard techniques. For example, since the host defense peptides are typically small, they can be conveniently synthesized chemically, by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like. Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The host defense peptides of the present invention can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

Alternatively, the host defense peptides can be produced by recombinant techniques. See, e.g., Zhang et al., FEBS Lett. (1998) 424:37-40; Zhang et al., J. Biol. Chem. (1999) 274:24031-24037; Shi et al., Infect. Immun. (1999) 67:3121-3127. The host defense peptides can be produced recombinantly, e.g., by obtaining a DNA molecule from a cDNA library or vector including the same, or from host tissue using phenol extraction. Alternatively, DNA encoding the desired host defense peptide can be synthesized, along with an ATG initiation codon. The nucleotide sequence can be designed with the appropriate codons for the particular amino acid sequence desired. In general, one selects preferred codons for the intended host in which the sequence is expressed. The complete sequence is generally assembled from overlapping oligonucleotides prepared by standard methods and assembled into a complete coding sequence. See, e.g., Edge Nature (1981) 292:756; Nambair et al. Science (1984) 223:1299; Jay et al. J. Biol. Chem. (1984) 259:6311. Automated synthetic techniques such as phosphoramide solid-phase synthesis, can be used to generate the nucleotide sequence. See, e.g., Beaucage, S. L. et al. Tet. Lett. (1981) 22:1859-1862; Matteucci, M. D. et al. J. Am. Chem. Soc. (1981) 103:3185-3191. Next the DNA is cloned into an appropriate vector, either procaryotic or eucaryotic, using conventional methods. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements. The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397. If present, the signal sequence can be the native leader found in association with the peptide of interest.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the peptides of interest. Mutants or analogs of host defense peptides for use in the subject compositions may be prepared by the deletion of a portion of the sequence encoding the molecule of interest, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known.

For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica.

Insect cells for use with baculovirus expression vectors include, inter alia, Aedes aegypti, Autographa californica, Bombyx mori, Drosophila melanogaster, Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

The host defense peptides, whether produced recombinantly or synthetically, are formulated into compositions and used in methods as detailed herein. Typical amounts of host defense peptides to be administered in the adjuvant compositions are from about 0.01 to about 8000 μg/kg, typically from about 0.05 to about 500 μg/kg, such as from 1 to 100 μg/kg, or 5 to 50 μg/kg, or any integer between these values.

Immunostimulatory Sequences

Bacterial DNA is known to stimulate mammalian immune responses. See, e.g., Krieg et al., Nature (1995) 374:546-549. This immunostimulatory ability has been attributed to the high frequency of immunostimulatory nucleic acid molecules (ISSs), such as unmethylated CpG dinucleotides present in bacterial DNA. Oligonucleotides containing unmethylated CpG motifs have been shown to induce activation of B cells, NK cells and antigen-presenting cells (APCs), such as monocytes and macrophages. See, e.g., U.S. Pat. No. 6,207,646, incorporated herein by reference in its entirety.

The present invention makes use of adjuvants that include components derived from ISSs. The ISS includes an oligonucleotide which can be part of a larger nucleotide construct such as plasmid or bacterial DNA. The oligonucleotide can be linearly or circularly configured, or can contain both linear and circular segments. The oligonucleotide may include modifications such as, but are not limited to, modifications of the 3′OH or 5′OH group, modifications of the nucleotide base, modifications of the sugar component, and modifications of the phosphate group. The ISS can comprise ribonucleotides (containing ribose as the only or principal sugar component), or deoxyribonucleotides (containing deoxyribose as the principal sugar component). Modified sugars or sugar analogs may also be incorporated in the oligonucleotide. Examples of sugar moieties that can be used include ribose, deoxyribose, pentose, deoxypentose, hexose, deoxyhexose, glucose, arabinose, xylose, lyxose, and a sugar analog cyclopentyl group. The sugar may be in pyranosyl or in a furanosyl form. A phosphorous derivative (or modified phosphate group) can be used and can be a monophosphate, diphosphate, triphosphate, alkylphosphate, alkanephosphate, phosphorothioate, phosphorodithioate, or the like. Nucleic acid bases that are incorporated in the oligonucleotide base of the ISS can be naturally occurring purine and pyrimidine bases, namely, uracil or thymine, cytosine, inosine, adenine and guanine, as well as naturally occurring and synthetic modifications of these bases. Moreover, a large number of non-natural nucleosides comprising various heterocyclic bases and various sugar moieties (and sugar analogs) are available, and known to those of skill in the art.

Structurally, the root oligonucleotide of the ISS can be a CG-containing nucleotide sequence, which may be palindromic. The cytosine may be methylated or unmethylated. Examples of particular ISS molecules for use in the present invention include CpG, CpY and CpR molecules, and the like, known in the art.

Such ISS molecules can be derived from the CpG family of molecules, such as CpG dinucleotides and synthetic oligonucleotides which comprise CpG motifs (see, e.g., Krieg et al. Nature (1995) 374:546 and Davis et al. J. Immunol. (1998) 160:870-876), any of the various immunostimulatory CpG oligonucleotides disclosed in U.S. Pat. Nos. 6,194,388; 6,207,646; 6,214,806; 6,218,371; 6,239,116; 6,339,068, US Publication No. 20030139364; PCT Publication No. WO 01/22990; PCT Publication No.; and WO 03/015711, all of which are incorporated herein by reference in their entireties. Such CpG oligonucleotides generally comprise at least 8 up to about 100 nucleotides, preferably 8 to 40 nucleotides, more preferably 15-35 nucleotides, preferably 15-25 nucleotides, and any number of nucleotides between these values. For example, oligonucleotides comprising the consensus CpG motif, represented by the formula 5′-X₁CGX₂-3′, where X₁ and X₂ are nucleotides and C is unmethylated, will find use as immunostimulatory CpG molecules. Generally, X₁ is A, G or T, and X₂ is C or T. Other useful CpG molecules include those captured by the formula 5′-X₁X₂CGX₃X₄, where X₁ and X₂ are a sequence such as GpT, GpG, GpA, ApA, ApT, ApG, CpT, CpA, CpG, TpA, TpT or TpG, and X₃ and X₄ are TpT, CpT, ApT, ApG, CpG, TpC, ApC, CpC, TpA, ApA, GpT, CpA, or TpG, wherein “p” signifies a phosphate bond. Typically, the oligonucleotides do not include a GCG sequence at or near the 5′- and/or 3′ terminus. Additionally, the CpG is usually flanked on its 5′-end with two purines (preferably a GpA dinucleotide) or with a purine and a pyrimidine (preferably, GpT), and flanked on its 3′-end with two pyrimidines, such as a TpT or TpC dinucleotide. Thus, molecules can comprise the sequence GACGTT, GACGTC, GTCGTT or GTCGCT, and these sequences can be flanked by several additional nucleotides, such as with 1-20 or more nucleotides, preferably 2 to 10 nucleotides and more preferably, 3 to 5 nucleotides, or any integer between these stated ranges. The nucleotides outside of the central core area appear to be extremely amendable to change.

Moreover, the ISS oligonucleotides for use herein may be double- or single-stranded. Double-stranded molecules are more stable in vivo while single-stranded molecules display enhanced immune activity. Additionally, the phosphate backbone may be modified, such as phosphorodithioate-modified, in order to enhance the immunostimulatory activity of the ISS molecule. As described in U.S. Pat. No. 6,207,646, CpG molecules with phosphorothioate backbones preferentially activate B-cells, while those having phosphodiester backbones preferentially activate monocytic (macrophages, dendritic cells and monocytes) and NK cells.

Different classes of CpG nucleic acids have been described. One class is potent for activating B cells but is relatively weak in inducing IFN-α and NK cell activation. This class has been termed the B class. The B class CpG nucleic acids are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068, incorporated herein by reference in their entireties. Another class is potent for inducing IFN-α and NK cell activation but is relatively weak at stimulating B cells; this class has been termed the A class. The A class CpG nucleic acids typically have stabilized poly-G sequences at 5′ and 3′ ends and a palindromic phosphodiester CpG dinucleotide-containing sequence of at least 6 nucleotides. See, for example, PCT Publication No. WO 01/22990, incorporated herein by reference in its entirety. Yet another class of CpG nucleic acids activates B cells and NK cells and induces IFN-α; this class has been termed the C-class. The C-class CpG nucleic acids typically are fully stabilized, include a B class-type sequence and a GC-rich palindrome or near-palindrome. This class has been described in PCT Publication No. WO 03/015711, the entire contents of which is incorporated herein by reference.

ISS molecules can readily be tested for their ability to stimulate an immune response using standard techniques, well known in the art. For example, the ability of the molecule to stimulate a humoral and/or cellular immune response is readily determined using the immunoassays described herein. Moreover, the adjuvant compositions and antigen can be administered with and without the ISS to determine whether an immune response is enhanced.

Exemplary, non-limiting examples of CpG oligonucleotides for use in the present compositions include those oligonucleotides 5′TCCATGACGTTCCTGACGTT3′ (SEQ ID NO:8), termed CpG ODN 1826, a Class B CpG; 5′TCGTCGTTGTCGTTTTGTCGTT3′ (SEQ ID NO:9), termed CpG ODN 2007, a Class B CpG; 5′TCGTCGTTTTGTCGTTTTGTCGTT3′ (SEQ ID NO:10), also termed CPG 7909 or 10103, a Class B CpG; 5′ GGGGACGACGTCGTGGGGGGG 3′ (SEQ ID NO:11), termed CpG 8954, a Class A CpG; and 5′TCGTCGTTTTCGGCGCGCGCCG 3′ (SEQ ID NO:12), also termed CpG 2395 or CpG 10101, a Class C CpG. All of the foregoing class B and C molecules are fully phosphorothioated.

Non-CpG oligonucleotides for use in the present composition include the double stranded polyriboinosinic acid:polyribocytidylic acid, also termed poly(I:C); and a non-CpG oligonucleotide 5′AAAAAAGGTACCTAAATAGTATGTTTCTGAAA3′ (SEQ ID NO:13).

Generally, the ISS present in the triple adjuvant composition will represent about 0.01 to about 1000 μg/kg, typically from about 0.05 to about 500 μg/kg, such as from 1 to 100 μg/kg, or 5 to 50 μg/kg, or any amount within these ranges, of the ISS per dose. One of skill in the art can determine the amount of ISS, as well as the ratio of ISS to the other components in the triple adjuvant composition.

Polyanionic Polymers

A polyanionic polymer of the present invention is a polymer which, when present in the triple adjuvant composition is negatively-charged due to the presence of anionic constitutional repeating units (for example, units containing sulphate, Y sulphonate, carboxylate, phosphate and borate groups). A constitutional repeating unit or I monomer refers to the minimal structural unit of a polymer. The polyanionic polymer may be a polyanionic heteropolymer, comprising two or more different anionic constitutional repeating units, or may be a polyanionic homopolymer, consisting of a single anionic constitutional repeating unit. Not every monomer/repeat unit need be negatively charged.

The polyanionic polymer for use in the adjuvant compositions may be a chemical polymer and may comprise anionic constitutional repeating units obtained from a group such as but not limited to acrylic acid, methacrylic acid, maleic acid, fumaric acid, ethylsulphonic acid, vinyl sulphuric acid, vinyl sulphonic acid, styrenesulphonic acid, vinylphenyl sulphuric I acid, 2-methacryloyloxyethane sulphonic acid, 3-methacryloyloxy-2 hydroxypropanesulphonic acid, 3-methacryl amido-3-methylbutanoic acid, acrylamidomethylpropanesulfonic acid, vinylphosphoric acid, 4-vinylbenzoic acid, 3 vinyl oxypropane-1-sulphonic acid, N-vinylsuccinimidic acid, and salts of the foregoing.

Alternatively, the polyanionic polymer used with the invention may be an oligo- or poly-saccharide such as dextran.

Additionally, the polyanionic polymer can be an oligopeptide or a polypeptide. Such peptides may be D- or L-peptides, and may comprise anionic constitutional repeating units (or monomers) such as L-aspartic acid, D-aspartic acid, L-glutamic acid, D-glutamic acid, non-natural anionic amino acids (or salts or anionic chemical derivatives thereof).

In certain embodiments, the polyanionic polymer may be a polymethyl methacrylate polymer, as well as a polymer derived from poly(lactides) and poly(lactide-co-glycolides), known as PLG. See, e.g., Jeffery et al., Pharm. Res. (1993) 10:362-368; and McGee et al., J. Microencap. (1996).

In some embodiments, the polyanionic polymer is a polyphosphazene. Polyphosphazenes are high-molecular weight, water-soluble polymers, containing a backbone of alternating phosphorous and nitrogen atoms and organic side groups attached at each phosphorus atom. See, e.g., Payne et al., Vaccine (1998) 16:92-98; Payne et al., Adv. Drug. Deliv. Rev. (1998) 31:185-196. Polyphosphazenes can form non-covalent complexes when mixed with compounds of interest, such as antigens and other adjuvants, increasing their stability and allowing for multimeric presentation. More than 700 polyphosphazenes are known with varying chemical and physical properties. For a review, see, Mark et al. in “Inorganic Polymers, 2nd Edition,” Oxford University Press, 2005. Typically, polyphosphazenes for use with the present triple adjuvant compositions will either take the form of a polymer in aqueous solution or a polymer microparticle, with or without encapsulated or adsorbed substances such as antigens or other adjuvants.

For example, the polyphosphazene component of the adjuvant compositions can be a soluble polyphosphazene, such as a polyphosphazene polyelectrolyte with ionized or ionizable pendant groups that contain, for example, carboxylic acid, sulfonic acid or hydroxyl moieties, and pendant groups that are susceptible to hydrolysis under conditions of use to impart biodegradable properties to the polymer. Such polyphosphazene polyelectrolytes are well known and described in, for example, U.S. Pat. Nos. 5,494,673; 5,562,909; 5,855,895; 6,015,563; and 6,261,573, incorporated herein by reference in their entireties.

Alternatively, polyphosphazene polymers in the form of cross-linked microparticles will also find use in the present adjuvant compositions. Such cross-linked polyphosphazene polymer microparticles are well known in the art and described in, e.g., U.S. Pat. Nos. 5,053,451; 5,149,543; 5,308,701; 5,494,682; 5,529,777; 5,807,757; 5,985,354; and 6,207,171, incorporated herein by reference in their entireties.

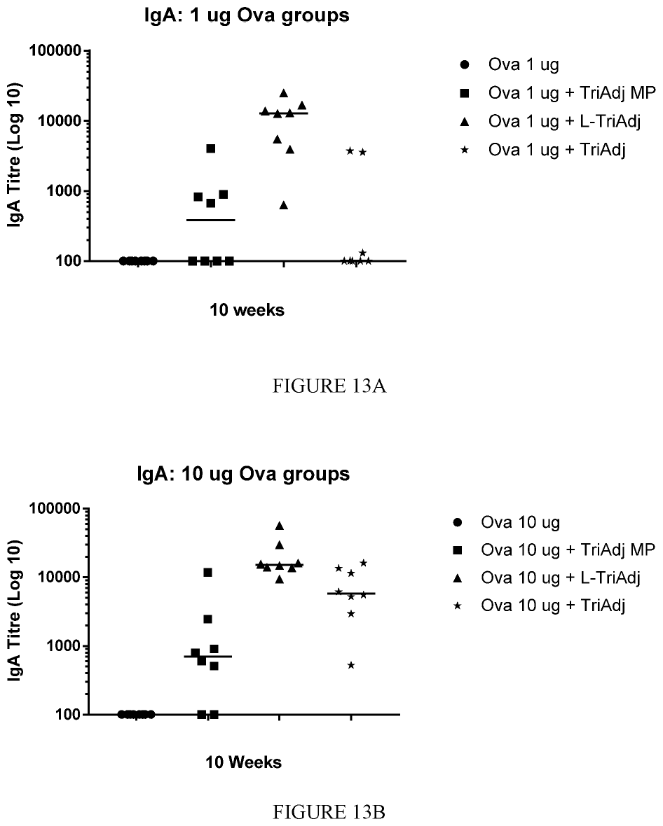

Exemplary polyphosphazene polymers for use in the present methods and triple adjuvant compositions are shown in FIG. 13 and include poly[di(sodium carboxylatophenoxy)phosphazene] (PCPP) and poly(di-4-oxyphenylproprionate)phosphazene (PCEP), in various forms, such as the sodium salt, or acidic forms, as well as a polymer composed of varying percentages of PCPP or PCEP copolymer with hydroxyl groups, such as 90:10 PCPP/OH. Methods for synthesizing these compounds are known and described in the patents referenced above, as well as in Andrianov et al., Biomacromolecules (2004) 5:1999; Andrianov et al., Macromolecules (2004) 37:414; Mutwiri et al., Vaccine (2007) 25:1204; and in U.S. Pat. Nos. 9,408,908 and 9,061,001, each of which is incorporated herein by reference in its entirety.

Typical amounts of polyphosphazene present in the triple adjuvant compositions will represent from about 0.01 to about 2500 μg/kg, typically from about 0.05 to about 500 μg/kg, such as from 0.5 to 100 μg/kg, or 1 to 50 μg/kg, or any amount within these values. One of skill in the art can determine the amount of polyphosphazene, as well as the ratio of polyphosphazene to the other components in the triple adjuvant composition.

Mucoadhesive Lipidic Carriers

The selected HDR, ISS and polyphosphazene are then combined to produce the triple adjuvant composition as described in the examples herein and in U.S. Pat. Nos. 9,408,908 and 9,061,001, each of which is incorporated herein by reference in its entirety. One of skill in the art can determine the ratio of the ISS:HDR:polyphosphazene present, which will depend on the particular components used. For example, in the case of poly(I:C)/IDR-1002/PCEP, the components can be present in a ratio of 1:2:1 (w/w/w). However, it is to be understood that this is just exemplary and other ratios will find use in the present compositions. This triple adjuvant composition is then combined with lipid components as described herein, to form positively charged mucoadhesive lipidic carrier systems, such as cationic liposomes encapsulating the adjuvant composition. The term “liposome” refers to vesicles comprised of one or more concentrically ordered lipid bilayers, which encapsulate an aqueous phase. The aqueous phase may contain the triple adjuvant composition and optionally the antigen to be delivered to the subject. The liposome ultimately becomes permeable and releases the encapsulated components mucosally. This can be accomplished, for example, in a passive manner wherein the liposome bilayer degrades over time through the action of various agents in the body. Alternatively, active agent release can be accomplished using an agent to induce a permeability change in the liposome vesicle. When liposomes are endocytosed by a target cell, for example, they alter the endosomal membrane and thereby cause release from the endosome. This destabilization is termed fusogenesis. 1,2-dioleoyl-sn-glycerol-3-phosphoethanolamine (DOPE) is the basis of many fusogenic systems.

In other embodiments, the cationic liposomes interact with the triple adjuvant composition by means of polyelectrolyte noncovalent attraction, resulting in a condensation reaction which generates nanoparticles. See, e.g., Bloomfield, V. A., Biopolymers (1991) 31:1471-1481; Bloomfield, V. A., Biopolymers (1997) 44:269-282; Morris et al., Curr. Opin. Biotechnol. (2000) 11:461-466; and Wadhwa et al., Bioconjug. Chem. (1997) 8:81-88. One of skill in the art can determine the ratio of the triple adjuvant ISS:HDR:polyphosphazene to the cationic liposomes, which will depend on the particular components used. The ratio of the components will determine if anionic, neutral or cationic lipid nanoparticle condensates are formed, with cationic lipid nanoparticles being preferred and shown in the examples. One of skill in the art can determine the ratio of the triple adjuvant ISS:HDR:polyphosphazene to the cationic liposomes which will affect the particle size of the condensed lipid nanoparticles. It is also possible to use lipids in the form of micelles, multilamellar vesicles, small unilamellar vesicles, large unilamellar vesicles, exosomes or in a solution in an organic solvent such as ethanol, methanol, chloroform, or the like.

Liposomes for use with the present invention can be unilamellar vesicles (possessing a single membrane bilayer) or multilameller vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Many methods exist for preparing liposomes and loading liposomes with therapeutic compounds. The simplest method of loading is by passive entrapment, wherein a dried lipid film is hydrated with an aqueous solution containing the water-soluble agent to form liposomes. Other passive entrapment methods involve a dehydration-rehydration method where preformed liposomes are added to an aqueous solution of the drug and the mixture is dehydrated either by lyophilization, evaporation, or by freeze-thaw processing that uses repeated freezing and thawing of multilamellar vesicles to improve hydration and hence increase loading. In order to improve entrapment efficiency, a high lipid concentration or specific combinations of lipid components can be used.

Thus, a variety of methods are available for preparing liposomes, such as, but not limited to sonication, extrusion, high pressure/homogenization, microfluidization, detergent dialysis, calcium-induced fusion of small liposome vesicles and ether-fusion methods, all of which are known to those of skill in the art. Methods for preparing liposomes are described in, e.g., Szoka et al., Ann. Rev. Biophys. Bioeng (1980) 9:467; U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, each of which is incorporated herein by reference in its entirety; PCT Publication No. WO 91\7424, incorporated herein by reference in its entirety; Deamer et al., Biochim. Biophys. Acta (1976) 443:629-634; Fraley, et al., Proc. Natl. Acad. Sci. USA (1979) 76:3348-3352; Hope et al., Biochim. Biophys. Acta (1985) 812:55-65; Mayer et al., Biochim. Biophys. Acta (1986) 858:161-168; Williams et al., Proc. Natl. Acad. Sci. USA (1988) 85:242-246; Liposomes (Ostro (ed.), Current Edition, Chapter 1); Hope et al., Chem. Phys. Lip. (1986) 40:89 (1986); Gregoriadis, Liposome Technology; and Lasic, Liposomes: from Physics to Applications.

Generally, particles are produced from materials that are non-reactive, biocompatible and available in pharmaceutical grade purity. The active agent(s) will be released in the body via particle degradation, erosion, swelling, or diffusion out of the matrix. As such, both the particle material as well as its degradation products, should be biocompatible. Furthermore, the particle material should be stable, able to efficiently encapsulate an optimal amount of active agent(s) and importantly, have the ability to contact the mucus layer covering the mucosal epithelial surface.

Various materials can be used to produce the cationic mucoadhesive particulate carrier systems, including cationic lipids such as, but not limited to, 1,2-dioleoyl-3-trimethylammonium-propane (DOTAP); 1,2-di-O-octadecenyl-3-trimethylammonium propane (chloride salt) (DOTAP); 3β-[N—(N′,N′-dimethylaminoethane)-carbamoyl] (DC); 3β-[N—(N′,N′-dimethylaminoethane)-carbamoyl]cholesterol hydrochloride (DC-Chol); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg or soy L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); ceramide carbamoyl-spermine (CCS); N⁴-Cholesteryl-Spermine HCL salt; and various combinations of one, two, three or more cationic lipids, such as more than one cationic lipid listed above; or lysolipid derivatives of cationic or phospholipids. Liposomes can also include various sugars, such as trehalose, e.g., trehalose 6,6,9-dibehenate (TDB), sucrose, lactose, mannitol or other common cryoprotectants and lyoprotectants known in the art.

Thus, for example, cationic liposomes can include DDAB and DOPE (DDAB/DOPE); DDAB, EPC and DOPE (DDAB/EPC/DOPE); SA and Chol (SA/Chol); EPC and Chol (EPC/Chol); SA, EPC and Chol (SA/EPC/Chol); DOTAP/DC/Chol; DDA and TDB (DDA/TDB); DSPC, TDB and DDA (DSPC/TBD/DDA); DMTAP and DMPC (DMTAP/DMPC), or any combination of cationic lipids so long as the liposomes retain the ability to contact the mucus layer. The above combinations are merely exemplary and other combinations can be determined by one of skill in the art.

When more than one cationic lipid is used, the components will be present in molar ratios that allow contact with the mucus layer and subsequent release of the liposome contents. Non-limiting examples of such ratios are for example, 50:50, 60:40, 75:25, or any integer within these ranges of DDAB:DOPE; 90:10; 80:20; 75:25, 70:30, or any integer within these ranges SA:Chol; 90:10; 80:20; 75:25, 70:30, or any integer within these ranges EPC:Chol; 40:50:10 DDAB:EPC:DOPE; and 40:50:10 SA/EPC/Chol. It is to be understood that these ratios can vary and the above amounts are exemplary only. One of skill in the art will be able to determine acceptable molar ratios for use with particular combinations.

Typically, for use in the present invention, the mean diameter of the mucoadhesive particles will be in the nanomeric range, such as from 1 nm to 1000 nm, e.g., 10 nm to 500 nm, 20 nm to 250 nm, such as under 300 . . . 250 . . . 200 . . . 150 . . . 100 . . . 50 nm, and so on. Particle size can be measured using any of various techniques, such as dynamic light scattering as described in the examples.

For a review of cationic liposome production and use for mucosal immunization, see, e.g., Chadwick et al., Advanced Drug Delivery Reviews (2010) 62:394-407; and Boddupalli et al., J. Adv. Pharm. Technol. Res. (2010) 1:381-387.

Vaccine Antigens

As explained above, the mucoadhesive carrier systems are able to be delivered to mucosa to enhance a local immune response, and in some cases systemic immunity, to a co-delivered vaccine antigen. An adjuvant composition comprising a host defense peptide, a polyphosphazene and an immunostimulatory sequence when delivered via a mucoadhesive lipidic carrier system as described herein, enhances the immune response vis-a-vis a selected antigen when it possesses a greater capacity to elicit a mucosal immune response than the immune response elicited by an equivalent amount of the antigen when delivered without the mucoadhesive lipid carrier system. Such enhanced immunogenicity can be determined by administering the antigen and the mucoadhesive lipid carrier system, and antigen controls to animals and comparing antibody titers against the two using standard assays such as radioimmunoassay and ELISAs, well known in the art.

Antigens for use with the adjuvant compositions include, but are not limited to, antigens of viral, bacterial, mycobacterial, fungal, or parasitic origin.

For example, the adjuvant compositions of the invention can be used in combination with antigens to treat or prevent a wide variety of infections caused by bacteria, including gram-negative and gram-positive bacteria. Particularly useful antigens for stimulating mucosal immunity will be derived from pathogens that invade the mucosa, such as, but not limited to pathogens that invade the respiratory tract, the GI tract, the urogenital tract and the eye.

Non-limiting examples of bacterial pathogens from which antigens can be derived include both gram negative and gram positive bacteria. Gram positive bacteria include, but are not limited to Pasteurella species, Staphylococci species, and Streptococcus species. Gram negative bacteria include, but are not limited to, Escherichia coli, Lawsonia intracellularis, Pseudomonas species, and Salmonella species. Specific examples of infectious bacteria include but are not limited to: Helicobacter pylori, Borelia burgdorferi, Legionella pneumophilia, Mycobacteria sp. (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sp.), Streptococcus pneumoniae, pathogenic Campylobacter spp., Enterococcus sp., Haemophilus infuenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

For example, the adjuvant compositions of the present invention can be used with any of the various Bordetella species including B. pertussis, B. parapertussis, B. bronhiseptica, and the like; various Neisserial species, including N. meningitidis, N. gonorrhoeae, etc.; various Enterobacteriaceae such as but not limited to Salmonella, such as S. typhimurium, S. enteritidis, Shigella, such as S. flexneri, Escherichia, such as E. coli 0157:H7, Klebsiella, Enterobacter, Serratia, Proteus, Morganella, Providencia, Yersinia, such as Y. enterocolitica, Listeria, such as L. monocytogene, Staphylococcus, such as S. aureus; various Pseudomonas species, such as P. aeruginosa; Stretococcal species, such as S. suis, S. uberis, S. agalactiae, S. dysgalactiae, S. pneumoniae, S. pyogenes, and the like; various Actinobacillus species, including but not limited to A. Pleuropneumoniae, A. suis, A. pyogenes, etc.

The adjuvant compositions can be used in combination with antigens to treat or prevent diseases caused by improper food handling, as well as diseases caused by food-borne pathogens, such as but not limited to Salmonella enteritidis, Salmonella typhimurium, Escherichia coli O157:H7, Yersinia enterocolitica, Shigella flexneri, Listeria monocytogene, and Staphylococcus aureus. Additionally, the adjuvant compositions are also useful in combination with antigens from pathogens that cause nosocomial infections, such as but not limited to pathogens that produce extended spectrum β-lactamases (ESBL) and thus have the ability to inactivate β-lactam antibiotics. These enzymes are produced by various bacteria, including Klebsiella pneumoniae, E. coli and Proteus mirabilis. Additionally, the adjuvant compositions can be used in combination with antigens to treat or prevent diseases caused by biocontamination of the skin by pathogenic microorganisms such as Staphylococcus aureus, S. epidermitidis, Pseudomonas aeruginosa, Acinetobacter spp., Klebsiella pneumoniae, Enterobacter cloacae, E. coli, Proteus spp. and fungi such as Candida albicans.

The adjuvant compositions can also be used in combination with antigens to treat or prevent respiratory conditions such as caused by Streptococcus pneumoniae, Haemophilus influenzae, and Pseudomonas aeruginosa, as well as sexually transmitted diseases, including but not limited to Chlamydia infections, such as caused by Chlamydia trachomatis and gonococcal infections, such as caused by Neisseria gonorrhoeae.

Additionally, the adjuvant compositions can be used with antigens to treat or prevent a number of viral diseases, such as but not limited to those diseases caused by members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; See, e.g. Virology, 3rd Edition (W. K. Joklik ed. 1988); Fundamental Virology, 2nd Edition (B. N. Fields and D. M. Knipe, eds. 1991), for a description of these and other viruses. Other particular examples of viruses include the herpesvirus family of viruses, for example bovine herpes virus (BHV) and human herpes simplex virus (HSV) types 1 and 2, such as BHV-1, BHV-2, HSV-1 and HSV-2, varicella zoster virus (VZV), Epstein-Barr virus (EBV), cytomegalovirus (CMV), HHV6 and HHV7; diseases caused by the various hepatitis viruses, such as HAV, HBV and HCV; diseases caused by papilloma viruses and rotaviruses, etc.

Non-limiting examples of viral pathogens that affect humans and/or nonhuman vertebrates from which antigens can be derived, or which can be provided in attenuated or inactivated form include retroviruses, RNA viruses and DNA viruses. The group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus, avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses from which antigens can be derived include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, BVDV, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus (BRSV), and Pneumonia virus of mice); forest virus; the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including the SARS virus, Infectious Bronchitis Virus (IBV), Mouse Hepatitis virus, Human enteric corona virus, Porcine epidemic diarrhea virus (PEDV) and Feline infectious peritonitis (Feline coronavirus). For example, for RSV vaccines, useful antigens include those derived from the fusion (F) protein, the attachment (G) protein, and/or the matrix (M) protein, or combinations thereof. These proteins are well known and can be obtained as described in U.S. Pat. No. 7,169,395, incorporated herein by reference in its entirety.

Illustrative DNA viruses from which antigens can be derived include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex virus Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, porcine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Similarly, the adjuvant compositions of the invention will find use against a variety of parasites, such as but not limited to Plasmodium, such as P. malariae, P. yoelii, P. falciparum, P. ovale, and P. vivax, Toxoplasma gondii, Schistosoma japonicum, Leishmania major, Trypanosoma cruzi, and so forth.

Additionally, the adjuvant compositions find use to enhance an immune response against a number of fungal pathogens, such as but not limited to those fungi causing Candidiasis, Cryptococcosis, Asperigillosis, Zygomycosis, Blastomycosis, Coccidioidomycosis, Histoplasmosis, Paracoccidiodomycosis, Sporotrichosis. Particular non-limiting examples of infectious fungi from which antigens can be derived include: Cryptococcus neoformans, Histoplasma capsulatum, Coccidioides immitis, Blastomyces dermatitidis, Chlamydia trachomatis, Candida albicans.

Other medically relevant microorganisms have been described extensively in the literature. See, e.g. C. G. A Thomas, Medical Microbiology, Bailliere Tindall, Great Britain 1983, the entire contents of which is hereby incorporated by reference.

Thus, it is readily apparent that the mucoadhesive lipidic carriers can be used in combination with a wide variety of antigens to enhance the immune response to prevent or treat diseases, such as infectious disease in humans, as well diseases in non-human animals.

These antigens can be provided as attenuated, inactivated or subunit vaccine compositions. Additionally, the antigens can be provided in nucleic acid constructs for DNA immunization. Techniques for preparing DNA antigens are well known in the art and described in, e.g., U.S. Pat. Nos. 5,399,346, 5,580,859, 5,589,466, incorporated by reference herein in their entireties.

The lipid encapsulated triple adjuvant compositions are also useful in combination with a number of commercial vaccines, in order to enhance a mucosal immune response to the co-delivered antigen. For example, the adjuvant compositions can be co-administered with commercially available human and animal vaccines, including but not limited to pertussis vaccines and combination vaccines, such as the various whole cell (wP) and acellular vaccines (aP). Nonlimiting examples of such vaccines include the vaccines known as TRIPEDIA, TRIPACEL, QUADRACEL, TETRAVAL, TETRACT-Hib, PENTACT-Hib, PENTACEL, PENTAVAC, and HEXAVAC (Aventis, Bridgewater, N.J.); INFANRIX and PEDIARIX (GlaxoSmithKline, Research Triangle Park, NC); CERTIVA (North American Vaccine, Beltsville, Md.); BIOTHRAX; TICE BCG; MYCOBAX; HiBTITER; PEDVAXHIB; ACTHIB; COMVAX; HAVRIX; VAQTA; TWINRIX; RECOMBIVAX HB; ENGERIX-B; FLUMIST; FLUVIDRIN; FLUZONE; JE-VAX; ATTENUVAX; M-M-VAX; M-M-R II; MENUMONE-A/C/Y/W-135; MUMPSVAX; PNEUMOVAX 23; PREVNAR; POLIOVAX; IPOL; IMOVAX; RABAVERT; MERUVAX II; DRYVAX; TYPHIM Vi; VIVOTIF; VARIVAX; YF-VAX.

The antigens/vaccines can be administered prior to, concurrently with, or subsequent to the lipid encapsulated triple adjuvant compositions. If administered concurrently, the antigens can be encapsulated or otherwise associated with the mucoadhesive lipid carrier or be delivered simultaneously in a separate formulation. If the lipid encapsulated triple adjuvant composition is administered prior to immunization with the antigen, it can be administered as early as 5-10 days prior to immunization, preferably 3-5 days prior to immunization and most preferably 1-3 or 2 days prior to immunization.

The antigens for use with the present invention can be prepared using standard techniques, well known in the art. For example, the antigens can be isolated directly from the organism of interest, or can be produced recombinantly or synthetically, using techniques described above.

Formulations and Administration

Some embodiments of the lipid encapsulated triple adjuvant composition and the antigen are formulated for delivery to mucosa, such as to the buccal cavity, sublingually, the nasal passages, the lungs, the GI tract, the eye, the urogenital tract, and the like. Thus, formulations include suppositories, aerosol, intranasal, oral formulations, and sustained release formulations. Methods of preparing such formulations are known in the art and described in, e.g., Remington's Pharmaceutical Sciences, Mack Publishing Company, Easton, Pa., Current edition.

Intranasal formulations will usually include pharmaceutically acceptable excipients that neither cause major irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa. Agents can be delivered intranasally using nasal drops, sprays, gels, suspensions and emulsions, an inhaler and/or an atomizer. Thus, the intranasal formulation may be administered by methods such as inhalation, spraying, liquid stream lavage, nebulizing, or nasal irrigation. The administering may be to the sinus cavity or the lungs.

For suppositories, the excipients will include traditional binders and carriers, such as, polyalkaline glycols, or triglycerides. Such suppositories may be formed from mixtures containing the active ingredient in the range of about 0.5% to about 10% (w/w), preferably about 1% to about 2%.

Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Aerosol delivery systems typically employ nebulizers and other inhaler devices and systems. Delivering drugs by inhalation requires a formulation that can be successfully aerosolized and a delivery system that produces a useful aerosol of the drug. The particles or droplets should be of sufficient size and mass to be carried to the distal lung or deposited on proximal airways to give rise to a therapeutic effect.

Some embodiments of the lipid encapsulated triple adjuvant composition and the antigen are formulated for delivery by injection to muscle tissue. Such embodiments may comprise pharmaceutically suitable excipients, diluents, and carriers. Examples of such pharmaceutically acceptable excipients, diluents, and carriers may be found in Remington: The Science and Practice of Pharmacy (2006). As well, examples of pharmaceutically acceptable carriers, diluents, and excipients may be found in, for example, Remington's Pharmaceutical Sciences (2000—20th edition) and in the United States Pharmacopeia: The National Formulary (USP 24 NF19) published in 1999, each of which are herein incorporated by reference in their entireties.

Vaccination is achieved in a single dose or repeated as necessary at intervals, as can be determined readily by one skilled in the art. For example, a priming dose can be followed by one or more booster doses at weekly, monthly, or longer intervals. An appropriate dose depends on various parameters including the recipient (e.g., adult or infant), the particular vaccine antigen, the route and frequency of administration, and the desired effect (e.g., protection and/or treatment), as can be determined by one skilled in the art. In general, the mucoadhesive lipidic carrier systems containing the triple adjuvant composition, and optionally a vaccine antigen, is administered by a mucosal route in an amount from 1 to 25 μg per kg.

Kits

The invention also provides kits. In certain embodiments, the kits of the invention comprise one or more containers comprising a mucoadhesive lipidic carrier that includes the triple adjuvant composition and optionally an antigen of interest, either encapsulated with the triple adjuvant composition, or in a separate container. The containers may be unit doses, bulk packages (e.g., multi-dose packages) or sub-unit doses.

In embodiments, the kits contain a mucosally acceptable excipient. The kits may comprise the components in any convenient, appropriate packaging. For example, if the mucoadhesive lipidic carrier systems are provided as a dry formulation (e.g., freeze dried or a dry powder), a vial with a resilient stopper can be used, so that the carrier may be resuspended by injecting fluid through the resilient stopper. Ampules with non-resilient, removable closures (e.g., sealed glass) or resilient stoppers can be used for liquid formulations. Also contemplated are packages for use in combination with a specific device, e.g., a nebulizer.

The kits can also comprise delivery devices suitable for mucosal delivery, such as an infusion device such as a minipump, an inhaler, and a nasal administration device (e.g., an atomizer).

The kits may further comprise a suitable set of instruction. The instructions generally include information as to dosage, dosing schedule, and route of administration for the intended method of use. Instructions supplied in the kits of the invention are typically written instructions on a label or package insert (e.g., a paper sheet included in the kit), but machine-readable instructions (e.g., instructions carried on a magnetic or optical storage disk) are also contemplated.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Materials Used in the Examples:

Polyinosinic-Polycytidylic acid (poly(I:C)) double-stranded RNA adjuvant (99% purity) was obtained from Sigma Aldrich (Canada). IDR-1002 cationic peptide adjuvant and poly(di-4-oxyphenylproprionate)phosphazene (a polyphosphazene known as PCEP), sodium salt (average molecular weight approximately 1800×10³) were used in the formulation. PCEP was obtained by custom synthesis at Idaho National Laboratory and can be prepared as described in U.S. Pat. Nos. 9,408,908 and 9,061,001, each of which is incorporated herein by reference in its entirety. The polyphosphazene tested endotoxin free.

IDR-1002 was obtained from Genscript (Piscataway Township, N.J.). The sequence of IDR-1002 was Val-Gln-Arg-Trp-Leu-Ile-Val-Trp-Arg-Ile-Arg-Lys-NH2 (SEQ ID NO:19).

Rhodamine-labeled poly(I:C) was from InvivoGen (San Diego, Calif. USA); agarose was from Invitrogen (Carlsbad, Calif. USA); gel loading dye 6× was from New England Biolabs Inc. (Ipswich, Mass., USA); and sterile syringe 0.2 μm filters were from Millipore.

Dimethyldioctadecylammonium bromide (DDAB) was from Sigma Aldrich (St. Louis, Mo., USA). Lipids 1,2-dioleoyl-sn-glycerol-3-phosphoethanolamine (DOPE) and egg L-αa-phosphatidylcholine (EPC) were from Avanti Polar Lipids (Alabaster, USA) and cholesterol was from J. T Baker (Center Valley, Pa. USA).

Cell line RAW 264.7 was obtained from the American Type Culture Collection (ATCC TIB-71™, Manassas Va. USA); MTS (tetrazolium compound [3-(4,5-dimethylthiazol-2-yl)-5-(3-carboxymethoxyphenyl)-2-(4-sulfophenyl)-2H-tetrazolium, inner salt; MTS) cell proliferation assay kit was from Promega (USA). Tissue culture medium Dulbecco's modified Eagle's medium (DMEM high glucose, GE Health Care, Canada) and 1% penicillin-streptomycin, were from Gibco, Canada. General chemicals Tris base, ethidium bromide, ascorbic acid, potassium phosphate monobasic, hydrochloric acid, boric acid and dextrose were from Sigma Aldrich (Canada). Porcine gastric mucin (Type II) and ovalbumin from chicken egg white (Ova) were from Sigma Aldrich (Canada).

### Example 1

### Preparation of the Triple Adjuvant Composition (TriAdj)

The triple adjuvant composition was prepared by mixing 150 μg of Poly(I:C), 300 μg of IDR-1002 peptide and 150 μg PCEP in 1:2:1 (w/w/w) ratio in a volume of 1 mL (see, Garg et al., J. Gen. Virol. (2014) 95:301-306). The diluent was sterile-filtered (0.2 μm) dextrose (5% (w/v) (D5W) and the preparation was carried out on ice and stored at 4° C. for use within 3 days (see, Garg et al., Hum. Vaccin. Immunother. (2017) 13:2894-2901). The formation of a non-dissociable complex was confirmed by agarose gel electrophoresis and fluorescence quenching that occurs upon interaction of the components.

### Example 2

### Preparation of Liposomes

Pre-formed liposomes were used for preparing a lipidic complex with the triple adjuvant composition, in order to readily control the proportions of lipid components, as well as the homogeneity of the mixture of lipids, while in the aqueous environment required for the triple adjuvant composition. The liposomes were prepared by the thin film extrusion method. Lipids at the appropriate molar ratios such as DDAB/DOPE 75:25; DDAB/DOPE 50:50; and DDAB/EPC/DOPE 40:50:10 were dissolved in chloroform. The various preparations were dried under a stream of filtered air to form a thin film in a glass tube. The thin film was dried under vacuum in a lyophilizer for 4-6 hours to remove the organic solvent. The dried lipid films were rehydrated using D5W. After hydration of the lipid films, the lipid suspensions were subjected to freeze-thaw 10 times resulting in formation of multilamellar vesicles (MLVs). The resulting preparation was extruded 10 times at 55-60° C. through polycarbonate filters (0.1 μm Whatman, Sigma Aldrich, St. Louis, USA) with an extruder apparatus (Lipex Extruder).

The mean diameter of the liposomes was determined by dynamic light scattering and zeta potential was measured in the D5W diluent, both at 23° C. (Nano ZS, Malvern Panalytical, Westborough, Mass.). Liposomal lipid concentration was quantified by a phosphorous assay described below.

### Example 3

### Phosphorous Assay

The total phosphorous (P) content was determined for the various liposomal formulations and for the triple adjuvant composition with different IDR-1002 peptide ratios. To do so, a modified version of the Fiske and Subbarow phosphorus assay was used. See, e.g., Chen et al., Anal. Chem. (1956) 28:1756-1758; Fiske et al., J. Biol. Chem. (1925) 66:374-389; and avantilipids.com/tech-support/analytical procedures/determination-of-total-phosphorus. Briefly, six standard solutions (containing 0 to 0.23 μmoles of phosphorus) were prepared in triplicate from a phosphorus standard solution (0.65 mM, Sigma Aldrich, St. Louis, Mo., USA), followed by addition of 0.45 mL of H₂SO₄ and heated in aluminum blocks at 200-215° C. for 25 minutes. The tubes were cooled for 5 minutes and 50 μl H₂O₂ was added, followed by heating at 200° C. for 30 minutes, to clarity. The tubes were cooled to ambient temperature, followed by addition of 3.9 mL deionized water, 0.5 mL ammonium molybdate tetrahydrate solution and 0.5 mL ascorbic acid solution.

Each tube was vortexed for 5 minutes before adding each solution. All the tubes were again heated to 100° C. for 7 minutes then cooled to ambient temperature. Absorbance was measured in triplicate at 820 nm in a spectrophotometer and phosphorous concentration calculated from the linear regression curve from the standards (r²>0.99).

### Example 4

### Preparation of Lipidic Triple Antigen Complexes (L-TriAdj)

The phosphorus (P) concentration was determined as described above. The molar ratio of P from the liposomes to P from the triple adjuvant composition was set as 0.5:1, 1:1, 2:1 and 3:1 to span a range of molar charge ratios (negative to positive), in order to determine empirically the composition necessary to achieve a cationic supramolecular assembly, i.e. positively charged lipidic nanoparticles. The goal was to establish component ratios that would facilitate favorable polyvalent polymer interactions between the cationic liposomes and the anionic triple adjuvant composition resulting in condensation (see, Bloomfield, V. A., Biopolymers (1997) 44:3) rather than gross aggregation. Liposomes and the triple adjuvant were separately diluted in D5W and subsequently consistent volume ratios of the two components were mixed to achieve different P molar ratios. The combination of liposomes and triple adjuvant to form the lipidic triple adjuvant complexes was performed by vortex mixing for 2 minutes followed by a 30 minute incubation at ambient temperature. The total P content was determined for the various liposome preparations and for the triple adjuvant composition. This information was used to devise molar ratios required to approximate the desired charge ratios of the lipidic complex of liposomes plus the triple adjuvant composition (L-TriAdj). The molar ratio of P from the liposomes to P from TriAdj was set as 0.5:1, 1:1, 2:1 and 3:1 (ratios 1, 2, 3 and 4, respectively).

### Example 5

### Preparation of CaCl₂ Microparticle Vaccines for In Vivo Studies

As a point of comparison, the triple adjuvant was prepared as microparticles (MPs) as previously described (see, Polewicz et al., Vaccine (2013) 31:3148-3155; Garlapati et al., Vaccine (2011) 29:6540-6548; Garlapati et al., Vaccine (2012) 30:5206-5214), without further characterization. Microparticles were prepared by a coacervation method, with poly(I:C) first mixed with IDR-1002 peptide at 37° C. for 30 minutes, and the PCEP and ovalbumin antigen separately combined. The poly(I:C)-peptide mixture was then combined with the PCEP and antigen mixture, followed by dropwise addition of 6.2% NaCl at a ratio of 1.95 mL of NaCl to 1 mL of 0.2% PCEP. The weight ratio of poly(I:C), IDR-1002 peptide and PECP was 10:20:10 μg. After 20 minutes at room temperature, 8% CaCl₂ solution was added to achieve a 1:200 dilution followed by 10 minute incubation at room temperature on a rocker. To collect the microparticles, the suspension was centrifuged at 1390×g for 10 minutes, washed with double-distilled H₂O and resuspended in phosphate-buffered saline. The pooled supernatants from these final steps were used to estimate ovalbumin antigen lost during formation of the microparticles. After filtering through 0.2 μm low protein binding syringe filters, typical encapsulation efficiency was approximately 70%.

### Example 6

### Particle Size and Zeta Potential Analysis

The average particle size (nm) and polydispersity index (PDI) of liposomes, the triple adjuvant composition (TriAdj) and cationic lipid-triple adjuvant nanoparticles (L-TriAdj) were determined by dynamic light scattering. Surface charge was estimated by zeta potential measurements (Nano ZS, Malvern Panalytical, Westborough, Mass.) in D5W at 23° C. Samples were measured in triplicate. Particle size and features were confirmed by scanning electron microscopy.

The mean diameter of all the liposome formulations was found to be <200 nm and for those containing DDAB, the zeta potential was highly positive. An excess of positive charge prevents particle aggregation by electrostatic repulsion. P ratios of 0.5:1 and 1:1 consistently resulted in gross visible aggregation and were not used further, likely representing samples with a net neutral surface charge. For L-TriAdj containing DDAB/DOPE (75/25) at 3:1 P ratio (ratio 4), aggregation was also observed and this composition was also eliminated. The in vivo studies described below utilized L-TriAdj prepared at 2:1 molar ratio of phosphorus (liposome:TriAdj), (ratio 3) as described above. L-TriAdj, DDAB/DOPE (50/50) produced particles that were smaller and more homogeneous than DDAB/DOPE (75/25) (see Tables 2 and 3). For this reason, the DDAB/DOPE (50/50) composition of L-TriAdj was used in the in vivo studies described below. The zeta potential of DDAB/DOPE 50/50 (mol/mol), DDAB/DOPE 75/25 and EggPC/chol 90/10 liposomes was 62.5, 78.6 and −5.89 mV, respectively. For L-TriAdj, the corresponding zeta potential values were reduced to 49.7, 56.4 and −18 mV, respectively, which were stable over 24 hours (FIG. 1). TriAdj content using weight ratios of 5:10:5, 6:25:12.5:6.25 or 12.5:25:12.5 (μg:μg:μg) of poly(I:C):IDR-1002 peptide:polyphosphazene did not significantly affect the particle size or zeta potential of L-TriAdj using these lipid formulations. The size analysis and zeta potential of L-TriAdj was assessed over 24 hours and found to be stable. For the whole vaccine of L-TriAdj and ovalbumin as administered to the mice for efficacy testing, the zeta potential was found to be stable for 24 hours at 1 μg of Ova mixed with L-TriAdj, but some polydispersity was noted at 24 hours when 10 μg ovalbumin was present.

TABLE 2
Size analysis and zeta potential of liposomes

| | Mean | | | | |
|---|---|---|---|---|---|
| Liposomal lipid | diameter | SD | | Zeta potential | |
| composition | (nm) | (nm) | PDI | (Mv) | SD |
| DDAB/DOPE | 128.2 | 67 | 0.1 | 62.5 | 5.2 |
| 50/50 | | | | | |
| DDAB/DOPE | 78 | 6.6 | 0.05 | 78.6 | 1.8 |
| 75/25 | | | | | |
| DDAB/EPC/Chol | 98 | 4.9 | 0.3 | 31.1 | 2.6 |
| 40/50/10 | | | | | |
| EPC/Chol 90/10 | 141 | 0.6 | 0.2 | −33.3 | 4.41 |

TABLE 3
Particle size analysis of the lipidic triple adjuvant particles
with multimodal distribution analysis of mean diameters

| Lipidic triple | | | | | | | | |
|---|---|---|---|---|---|---|---|---|
| adjuvant | 0 hours | | 1 hour | | 6 hours | | 24 hours | |
| particles | Peak1 | Peak2 | Peak1 | Peak2 | Peak1 | Peak2 | Peak1 | Peak2 |
| DDAB/DOPE | 140 | | 146.8 | | 48.7 | | 41.3 | |
| 50/50 ratio3 | | | | | | | | |
| DDAB/DOPE | 96.3 | | 91.4 | | 9.2 | | 9.2 | |
| 50/50 ratio4 | | | | | | | | |
| DDAB/DOPE | 105.9 | 297.7 | 98.6 | 317 | 117.9 | 256 | 114.8 | 252.5 |
| 75/25 ratio3 | | | | | | | | |
| DDAB/DOPE | 139.9 | 471.8 | 146 | | 152 | | 105 | 295 |
| 75/25 ratio4 | | | | | | | | |

### Example 7

### Mucin Binding Studies

Mucin in deionized water (5 mg/mL) was freshly prepared prior to each experiment. The mixture of L-TriAdj or liposomes with mucin was incubated for 30 minutes at ambient temperature and mixed by vortex immediately prior to particle sizing and zeta potential analysis, performed at 23° C. (Nano ZS, Malvern Panalytical, Westborough, Mass.). Samples were measured in triplicate. Multimodal analysis with number weighting was used for the particle sizing.

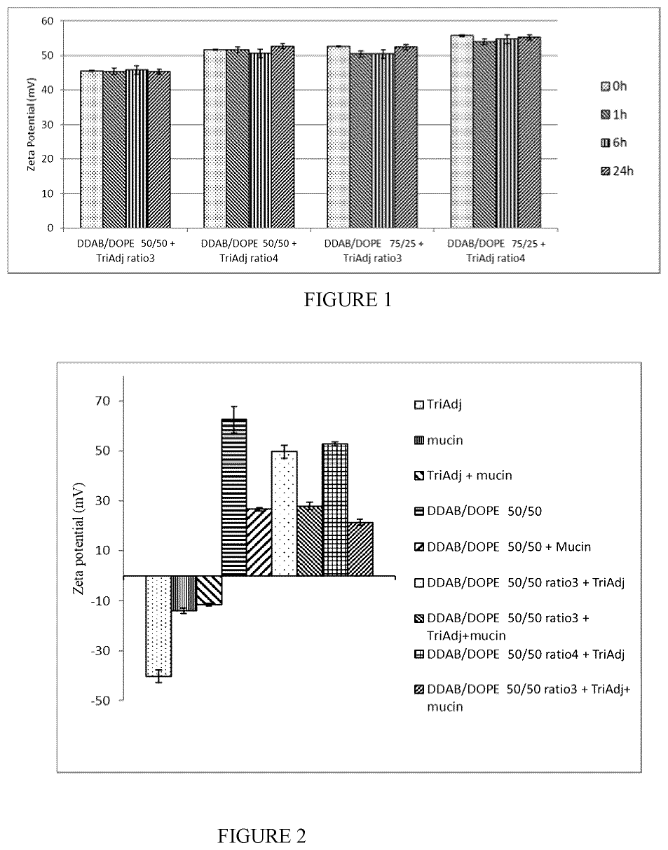

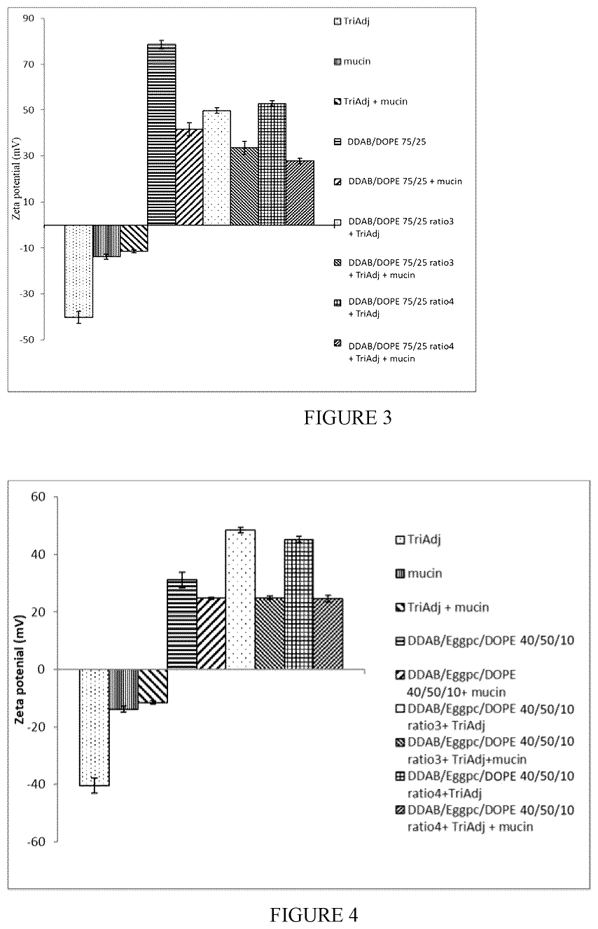

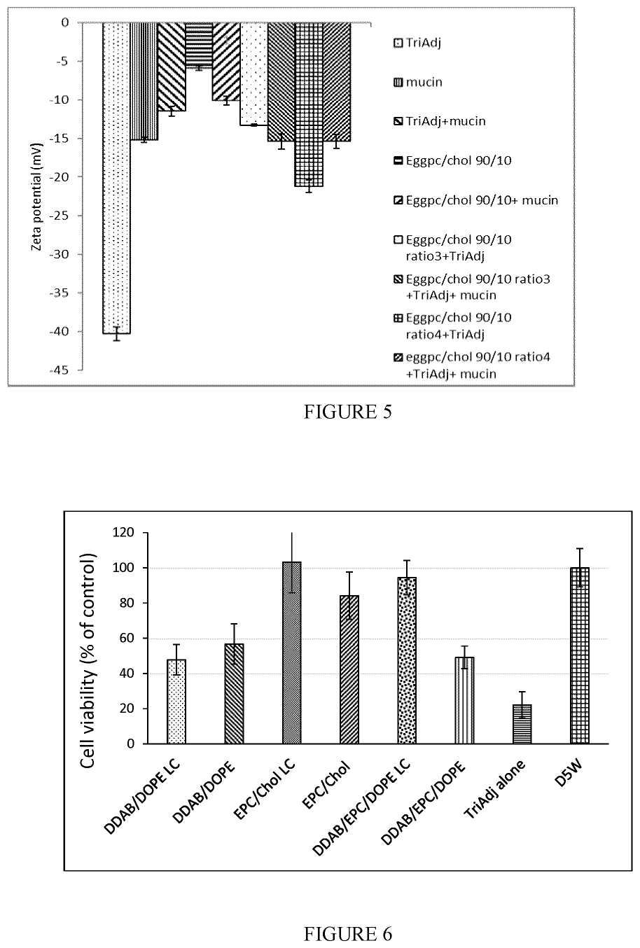

To assess the potential for mucoadhesion, the zeta potential of liposomes, TriAdj and L-TriAdj was measured before and after addition of mucin (5 mg/ml). Zeta potential is a measurement of the electrical potential difference between the particle surface and the bulk liquid phase. Here, a change in zeta potential was used as a surrogate measure of mucin binding as the zeta potential value will change if mucin adsorbs or binds to the particle surface. It does not reflect the affinity nor the specificity of binding. FIGS. 2 to 4 show that cationic liposomes alone and in association with the triple adjuvant compositions, bind to mucin. Cationic liposomes alone composed as DDAB/DOPE 50/50 (FIG. 2), DDAB/DOPE 75/25 (FIG. 3), and DDAB/EPC/DOPE 40/50/10 (FIG. 4) showed initial zeta potential values of 62.5, 78.6 and 31 mV, respectively, which decreased significantly upon addition of TriAdj (forming L-TriAdj). This suggests that the liposomes formed a complex with the triple adjuvant composition. The triple adjuvant composition alone had a negative zeta potential (−45 mV). When mucin was added to L-TriAdj, the zeta potential further decreased, consistent with an interaction. EPC/Chol 90/10 (FIG. 5) was used as a negative control, and showed a slight change in the zeta potential of the liposomes (−33 mV) when mixed with TriAdj and mucin, suggesting nonspecific interactions.

### Example 8

### Cytotoxicity Assay

Cytotoxicity or the triple adjuvant composition versus the L-TriAdj was assessed in a mouse macrophage cell line, RAW 267.4, by an MTS assay. Cells were cultivated in DMEM (Dulbecco's modified Eagle's medium) high glucose (10% FBS, 1% antibiotics (1% penicillin-streptomycin)), at 37° C. and 5% CO₂. Cells were seeded as 5,000 cells/well in 96-well plates and allowed to adhere for 24 hours. Cells were then treated with the triple adjuvant composition or lipidic triple adjuvant particles comprised of DDAB/DOPE 60/40 or DDAB/EPC/DOPE (45/45/10) as the lipid component and incubated at 37° C. for 24 hours. After 24 hours, 20 μL of CellTiter 96® Aqueous One Solution Reagent (Promega, Madison Wis.) was added into each well of the 96-well plate. After 3 hours of incubation, the absorbance at 490 nm was measured using a Biotek Synergy HT Microplate Reader™ (BioTek, Winooski, Vt.). The vehicle control was D5W and wells with only culture medium were used as a background. One-way ANOVA with Tukey's post-hoc test was used to determine significant differences (n=4, p<0.05).

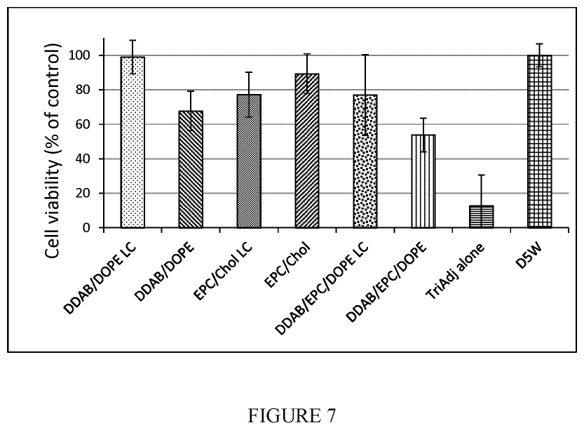

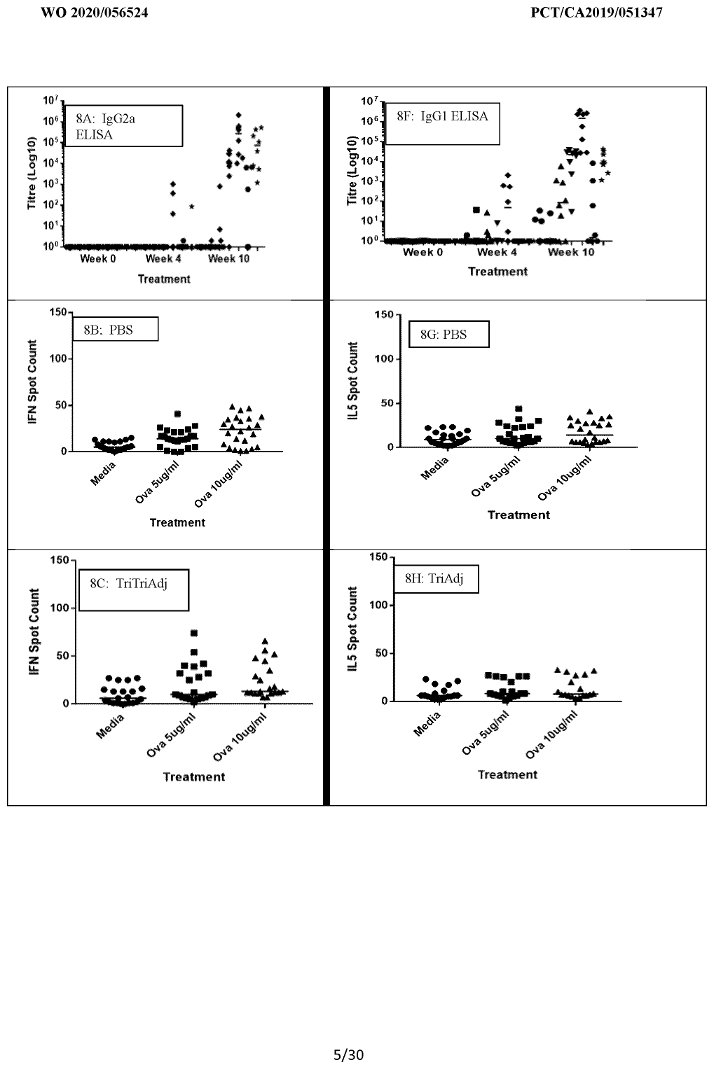

In the MTS assay, TriAdj content was constant at 0.5:1:0.5 (μg:μg:μg)/well (FIG. 6) and 0.25:0.5:0.25 μg:μg:μg/well (FIG. 7). The triple adjuvant composition alone was significantly more toxic (p<0.01) compared to liposomes comprised of DDAB/DOPE (50:50 mol:mol); EPC/Chol (90:10); DDAB/EPC/DOPE (40:50:10), or as L-TriAdj lipid complexes (LC) (ratio 3).

### Example 9

### In Vivo Studies: Intranasal Vaccination in Mice

To assess the adjuvant activity of the lipidic triple adjuvant particles, three in vivo studies were conducted with intranasal administration of an ovalbumain (Ova) vaccine in mice. The first study compared two different lipid compositions of L-TriAdj as well as 2 different doses of TriAdj with a constant weight ratio of polyphosphazene:peptide:poly(I:C), i.e. 1:2:1 or 5:10:5 (μg:μg:μg). Balb/c mice were randomly divided into 7 adjuvant groups (n=8/group). The mice were also randomized to cages such that the various treatment groups were not together in the same cage. All groups, except PBS control and Ova control, received 1 μg Ova antigen mixed with the adjuvant just prior to intranasal administration (20 μL; 10 μL/nostril). Treatment Groups: A: PBS control (no vaccine); B: Ova control (1 μg) (antigen only, no adjuvant); Groups C-G received Ova antigen along with the indicated adjuvant: C: TriAdj (5:10:5); D: L-TriAdj as DDAB/DOPE 60/40 (mol/mol) (TriAdj 1:2:1); E: L-TriAdj as DDAB/DOPE 60/40 (TriAdj 5:10:5); F: L-TriAdj as DDAB/EPC/DOPE 45/45/10 (TriAdj 1:2:1); G: L TriAdj as DDAB/EPC/DOPE 45/45/10 (TriAdj 5:10:5).

In the second study, a comparison of L-TriAdj coformulated with the ovalbumin antigen versus a calcium microparticle formulation of TriAdj (MP, see above) was performed in a similar way as described above. In this second study, the dose of antigen was varied as 1 μg or 10 μg with either no adjuvant, TriAdj mixed with antigen, TriAdj coformulated with the antigen in calcium chloride microparticles, or L-TriAdj mixed with antigen. All mice received 20 μL intranasally as in the first study. The treatment groups (n=8 mice/group) were: A: Ova control (1 μg) (antigen only, no adjuvant); B: Ova control (10 μg) (antigen only, no adjuvant); Groups C-G all received the triple adjuvant as the 5:10:5 ratio of poly(I:C):IDR-1002 peptide:polyphosphazene but as the following formulations: C: Ova 1 μg coformulated in the microparticle adjuvant; D: Ova 10 μg coformulated in the microparticle adjuvant; E: Ova 1 μg+L-TriAdj DDAB/DOPE (50/50 mol/mol); F: Ova 10 μg+L-TriAdj DDAB/DOPE (50/50 mol/mol); G: Ova 1 μg+TriAdj; H: Ova 10 μg+TriAdj.

In the third study, a comparison of the intranasal and the intramuscular routes of administration for both L-TriAdj coformulated with the ovalbumin antigen and a calcium microparticle formulation of TriAdj (MP, see above) was performed in a similar way as described above. In this third study, the dose of antigen was 10 μg either with no adjuvant or mixed with TriAdj coformulated with the antigen in calcium chloride microparticles or with L-TriAdj. Mice administered intranasally received 20 μL as in the first and second studies; mice administered intramuscularly received 50 μL (25 μL/leg). The treatment groups (n=8 mice/group) were: A: Ova 10 μg+L-TriAdj DDAB/DOPE (50/50 mol/mol) delivered intranasally in 20 μL; B: Ova 10 μg+L-TriAdj DDAB/DOPE (50/50 mol/mol) delivered intramuscularly in 50 μL; C: Ova 10 μg coformulated in the microparticle adjuvant, delivered intranasally in 20 μL; D: Ova 10 μg coformulated in the microparticle adjuvant, delivered intramuscularly in 50 μL; E: Ova control (10 μg) (antigen only, no adjuvant), delivered intramuscularly in 50 μL. Groups A-D all received TriAdj as the 5:10:5 ratio of poly(I:C):IDR-1002 peptide:polyphosphazene.

In all three studies, the mice were vaccinated at day 0 and day 28 with the same dose. Serum was collected on days 0, 14, 28, 42, 56, and 70 for IgG1 and IgG2a ELISAs, as well as for IgA ELISAs for the second study (Week 10 only) and the third study. Mice were euthanized and spleens were collected on days 70 and 72 (note for the third study: two mice of each group were euthanized at day 70, three at day 72 and the last three at day 73; results did not show an effect of euthanasia day). Each spleen was used for lymphocyte activation assays by ELISpot assay, described below. The analyst was blinded to treatment group during the ELISA and ELISpot assays.

To measure antigen-specific IgG1, IgG2a and IgA serum levels post vaccination, serum was collected from mice at 0, 2, 4, 6, 8 and 10 weeks. ELISAs were performed on the collected sera as previously described. See, Garg et al., Vaccine (2015) 33:1338-1344. Plates were coated overnight with ovalbumin at 4° C. and incubated with sera diluted 100:800. To detect IgG1, IgG2a and IgA, biotin-labeled goat anti-mouse IgG1, IgG2a or IgA was added (IgG1: Invitrogen Cat. No. A10519; IgG2a: Invitrogen Cat. No. M32315; IgA: Invitrogen Cat. No. M31115) followed by streptavidin-alkaline phosphatase (AP) (016-050-084, Jackson ImmunoResearch Laboratories Inc., West Grove, Pa.). A colorimetric reaction was developed using p-nitrophenyl phosphate (Sigma-Aldrich, St. Louis, Mo.) as the AP substrate. Plates were read with a Biorad iMark Microplate Reader™. Data were expressed as titres, which represent the dilution factor required to generate an absorbance reading three standard deviations above the average value of the negative control, e.g. serum from control mice receiving no vaccination.

To measure antigen-specific IgG1, IgG2a and IgA levels from bronchioalveolar lavages (BALs) and intranasal washes, these samples were collected on mice at 10 weeks at the time of euthanasia.

For ELISpot assays, spleens were harvested from mice at time of euthanasia and placed in 10 mL Minimal Essential Medium (MEM, Gibco, Canada) on ice. The spleens were sieved through a 40 μm strainer (BD Falcon) and the cells pelleted at 1000 rpm for 10 minutes at 4° C. The cell pellet was resuspended in 5 mL Gey solution and incubated at room temperature for 10 minutes. 9 mL MEM was added to this solution and followed by centrifugation twice as described above. The final pellet was resuspended in 2 mL AIM V media (Gibco) and the cells counted using the Millipore Scepter handheld automated cell counter. ELISpot assays were performed as described previously. See, Garlapati et al., Vaccine (2011) 29:6540-6548; Garg et al., Vaccine (2015) 33:1338-1344; Garg et al., Virology (2016) 499:288-297. Briefly, ELISpot plates (Millipore, Billerica, Mass., USA) were coated overnight with IL5 or IFN-γ at 2 μg/mL (BD Biosciences Cat. No. 551216 and 554393). Spleen samples were then added in triplicate at a concentration of 1×10⁷ cells/mL and incubated overnight. Splenocytes were stimulated with two different concentrations of ovalbumin: 5 μg/mL and 10 μg/mL. Spots representing IFN-γ- or IL-5-secreting cells were developed with biotinylated IFN-γ- or IL-5-specific goat anti-mouse IgG (BD Biosciences, 554410, 554397), followed by AP-conjugated streptavidin and BCIP/NBT (Sigma-Aldrich, B5655) as the substrate. Spots were counted with an AID ELISpot Reader (Autoimmun Diagnostika GmbH, Germany).

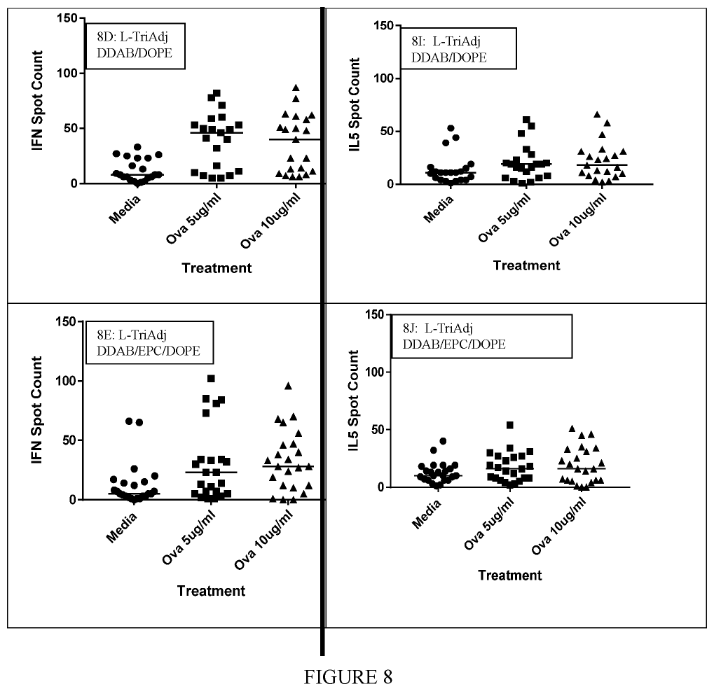

The results obtained from the first in vivo study in mice are shown in FIGS. 8A-8J and show a significantly greater response with the lipid-based complex following intranasal administration with the lower dose of ovalbumin antigen (Ova) compared to the triple adjuvant composition alone (TriAdj). At a higher dose of Ova, both groups performed equally well. As explained above, in order to assess humoral (Th2 type) versus cellular (Th1 type) immune responses to vaccination, serum levels of IgG1 and IgG2a were measured at 0, 4 and 10 weeks by ELISA (FIGS. 8A and 8F). L-TriAdj comprised of DDAB/DOPE with TriAdj at 5:10:5 weight ratio of poly(I:C):IDR-1002 peptide:polyphosphazene (μg:μg:μg) generated significantly higher IgG1 levels compared to TriAdj alone (p<0.01) but this was not the case for DDAB/EPC/DOPE at either amount of TriAdj. Rank-order transformation of the IgG1 titer values revealed that groups receiving L-TriAdj based on DDAB/DOPE at both doses of TriAdj (1:2:1 and 5:10:5) or DDAB/EPC/DOPE formulated at 5:10:5 weight ratio, were statistically significantly higher (p<0.01) than from groups receiving TriAdj at 5:10:5 weight ratio. Comparison of the rank order data further showed a significant difference in IgG1 response between mice receiving L-TriAdj at 1:2:1 versus 5:10:5 weight ratios of TriAdj (p<0.05). Furthermore, the median IgG2a responses of mice in groups receiving the lipid formulations were significantly higher than those receiving TriAdj alone as the adjuvant, as shown in FIG. 8F. There were significant differences between the rank-order transformed IgG2a values from groups receiving doses of TriAdj at 1:2:1 vs. 5:10:5 ratios for both DDAB/DOPE and DDAB/EPC/DOPE-based L-TriAdj (p<0.01). However, there was no statistically significant difference in IgG2a response comparing the two lipid-based adjuvants at the 5:10:5 ratio at week 10.

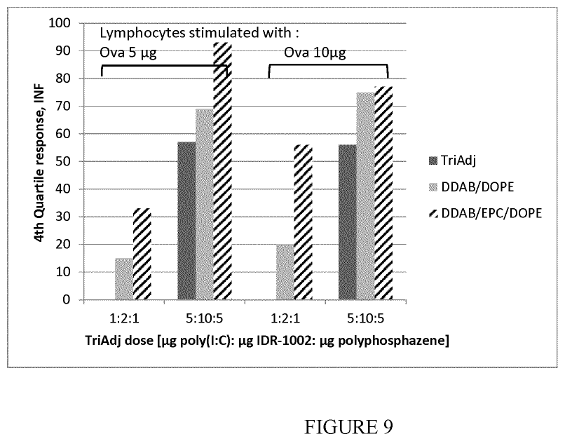

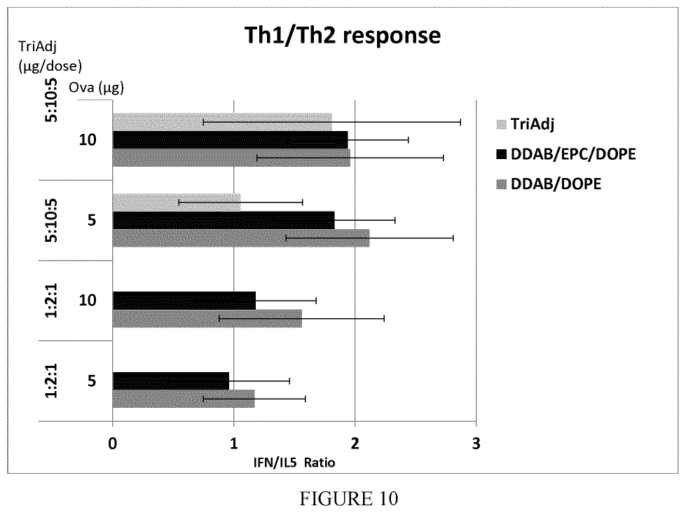

Lymphocytes were isolated from the spleens of vaccinated mice and their response to the ovalbumin antigen was assessed ex vivo by measurement of secreted IFN-γ and IL-5 (ELISPOT assay). FIG. 8 is organized with the left side representing the cellular (Th1) response (IgG2a and IFN-γ) and the right side representing the humoral (Th2) response (IgG1 and IL-5). A balanced Th1/Th2 response is desirable and a Th1 type response is essential for vaccines intended for viral infections in order to promote cytotoxic killing of infected cells. Secretion of IL-5 from lymphocytes obtained from the vaccinated mice was not significantly different between the various treatment groups (FIGS. 8G, 8H, 8I and 8J). However, ELISpot results for secretion of IFN-γ from Ova-stimulated splenocytes (FIGS. 8B, 8C, 8D, 8E) showed a greater proportion of strong responders in the groups vaccinated with L-TriAdj at the 5:10:5 weight ratio compared to TriAdj alone as the adjuvant. This dose-response to the triple adjuvant content within L-TriAdj is illustrated in FIG. 9, where lymphocytes from vaccinated mice stimulated with a recall dose of 5 or 10 μg/mL Ova showed a higher level of IFN-γ release for those groups that received L-TriAdj at 5:10:5 weight ratio of the adjuvant. FIG. 10 represents an analysis of the polarization of the T cell response relative to lipid composition, adjuvant dose and Ova antigen dose. A value <1 implies a relatively greater Th1 type response. A value >1 implies a stronger Th2 response. With both TriAdj and L-TriAdj, a desirable balanced response was noted.

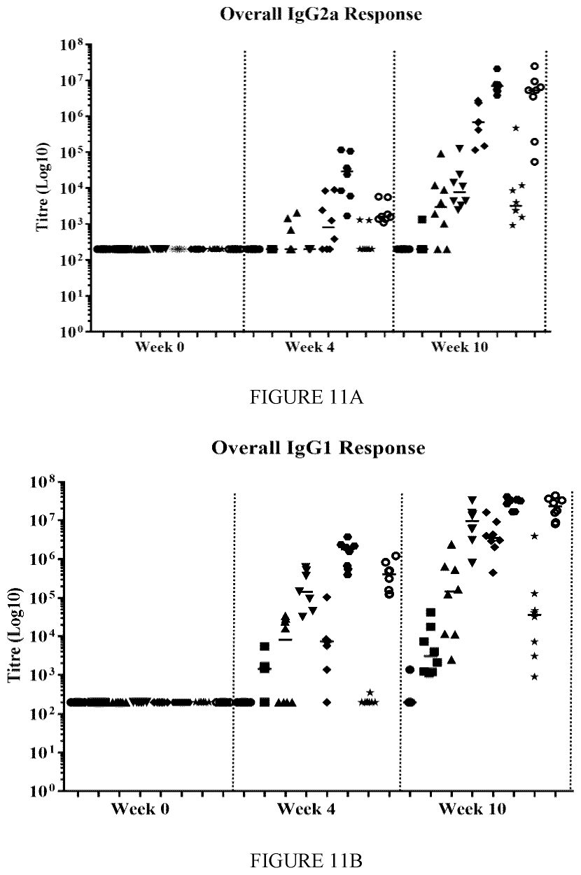

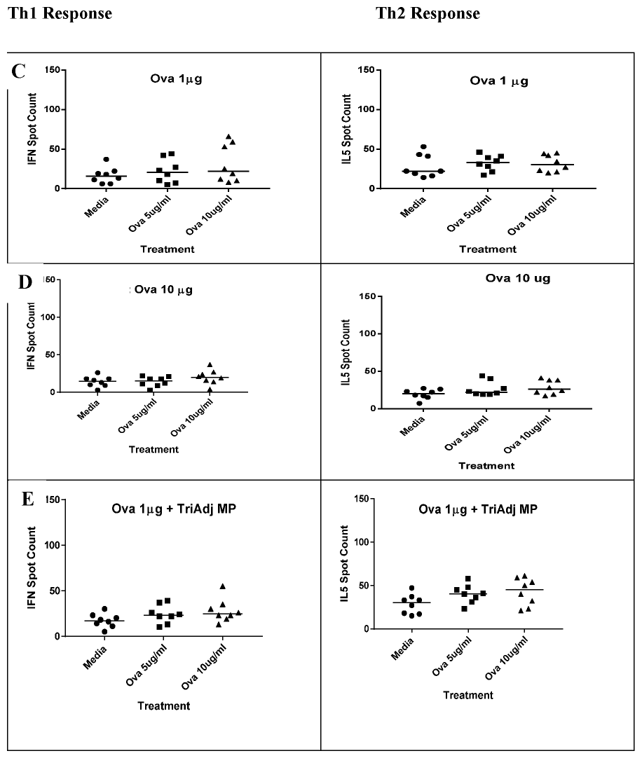

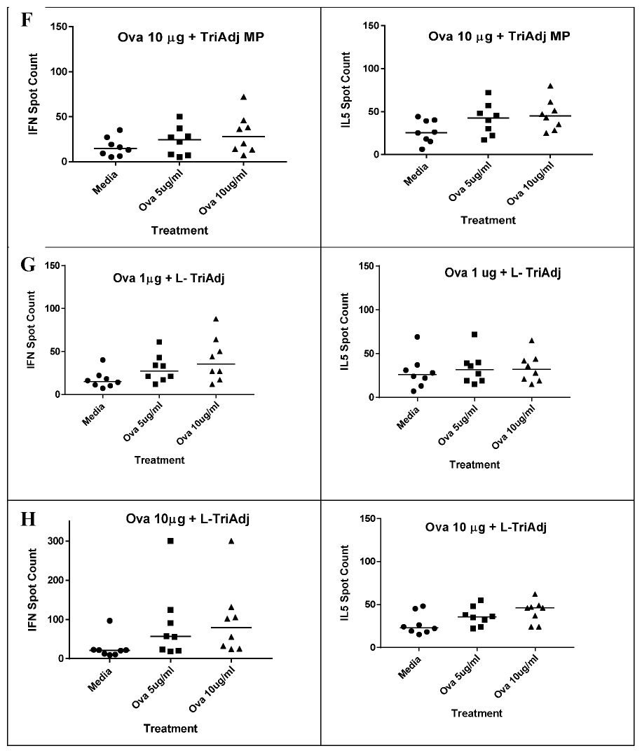

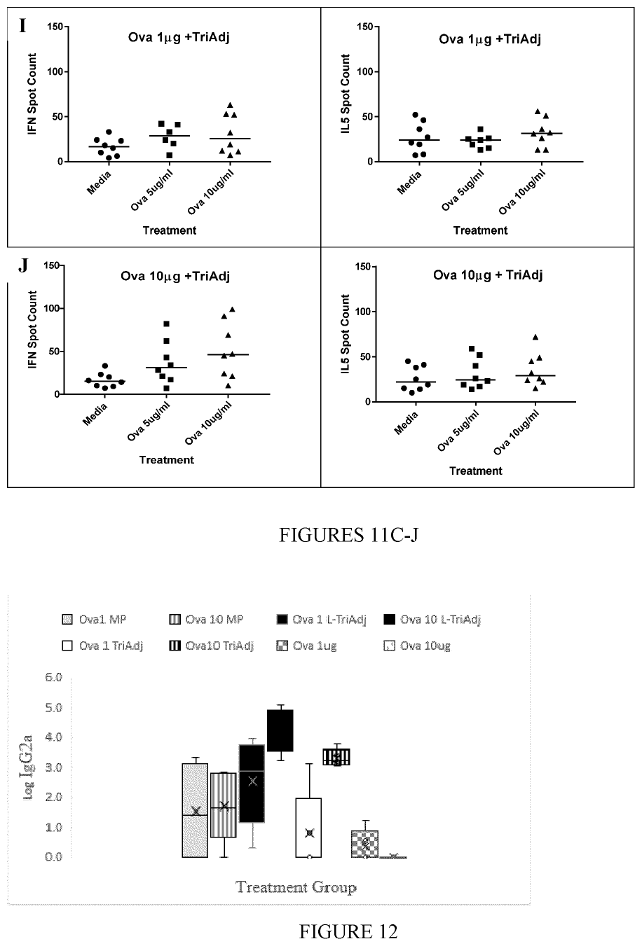

FIGS. 11A-11J show the results of the second in vivo study in mice, comparing the adjuvant ability of TriAdj formulated as calcium microparticles versus L-TriAdj or TriAdj alone. FIG. 11A represents the serum IgG2a levels at 0, 4, and 10 weeks from mice receiving intranasal Ova vaccines adjuvanted with TriAdj, TriAdj microparticles, or L-TriAdj, as measured by ELISA assay. PBS and Ova without adjuvant served as controls. The Ova antigen dose was varied as 1 or 10 μg/dose for all adjuvant and control groups. A booster dose was administered intranasally at week 4.

FIG. 11B represents the corresponding IgG1 serum levels from the same animals. At 4 weeks, for the microparticle and lipidic formulations of TriAdj, the IgG1 titres were similar for mice vaccinated with 1 versus 10 μg Ova, and a similar trend was seen with IgG2a titres. However, the soluble TriAdj required 10 μg Ova to generate IgG1 and IgG2a titres comparable to that achieved with 1 μg Ova with L-TriAdj as the adjuvant. At 4 weeks, the microparticle formulation of TriAdj with 1 μg Ova generated lower IgG2a titres compared to L-TriAdj with 1 μg Ova, whereas the IgG1 titres were similar for the same antigen dose (1 or 10 μg Ova). The titres at 10 weeks from vaccinated mice were higher than at 4 weeks. At the high dose of antigen (10 μg Ova), there was no significant difference in IgG1 titres between groups receiving the vaccine adjuvanted with TriAdj, microparticle TriAdj or L-TriAdj, however in observing the IgG2a titres, it can be seen that the microparticle formulation induced a lower titre than the other two adjuvant groups at 10 μg Ova/dose. Furthermore, L-TriAdj outperformed the other adjuvants at an Ova dose of 1 μg in terms of IgG2a response, demonstrating its potential for an antigen dose-sparing effect.

FIGS. 11C-11J represent the IFN-γ (left-side) and IL-5 response (right side) from lymphocytes obtained from the spleens of the vaccinated mice, following ex vivo stimulation with Ova antigen at 5 or 10 μg/mL, as measured by ELISpot assay. Thus, the effect not only of adjuvant formulation type and antigen dose, but also the range of response to antigenic recall at two doses, was compared. FIGS. 11C and 11D illustrate the response of lymphocytes from mice vaccinated with Ova alone (no adjuvant) at 1 or 10 μg/dose, respectively. Within each formulation group and antigen dose, the median response of the lymphocytes to the Ova recall was similar at 5 versus 10 μg/mL Ova for both the IFN-γ and IL-5 ELISpot results and for the L-TriAdj and microparticle adjuvant groups, however, a greater response was noted in IL-5 and IFN-γ values when 10 μg Ova antigen was included in the vaccine (FIGS. 11F, 11H, 11J) compared to 1 μg Ova (FIGS. 11E, 11G, 11I). Similar IL-5 and IFN-γ values were measured from groups receiving L-TriAdj as the adjuvant and 1 μg Ova compared to the microparticle formulation of TriAdj and 10 μg of Ova in the vaccine.

From these results, at least three key features are notable: a potentially reduced antigen dose requirement (antigen sparing) with L-TriAdj (DDAB/DOPE 50:50) as observed at 10 weeks; an earlier immune response for mice receiving L-TriAdj (10 μg Ova); and the maintenance of a balanced Th1/Th2 immunity. This difference between formulations is less evident at the higher dose of antigen, which generated measurable IgG1 and IgG2a responses even without an adjuvant as shown in FIGS. 11A and 11B. In comparing the immune response at 4 weeks, before the booster dose, the median IgG2a response for L-TriAdj was significantly greater than that of the microparticle TriAdj formulation or TriAdj at the 10 μg dose of Ova antigen. FIG. 12 illustrates the median IgG2a titres for these groups at 4 weeks. Consistent with the first in vivo study using the lipid formulation of TriAdj, the IgG2a antibody titres and INF-γ secretion from lymphocytes of vaccinated mice indicate a strong cell-mediated response for both the lipidic and microparticle formulations.

Still in this second mouse study, serum levels of IgA, a marker of mucosal immunity, were measured at 10 weeks (FIG. 13). All adjuvanted administrations of 10 g Ova induced significantly higher levels of IgA than the administration of ovalbumin alone, with L-TriAdj formulation showing the highest levels (FIG. 13B).

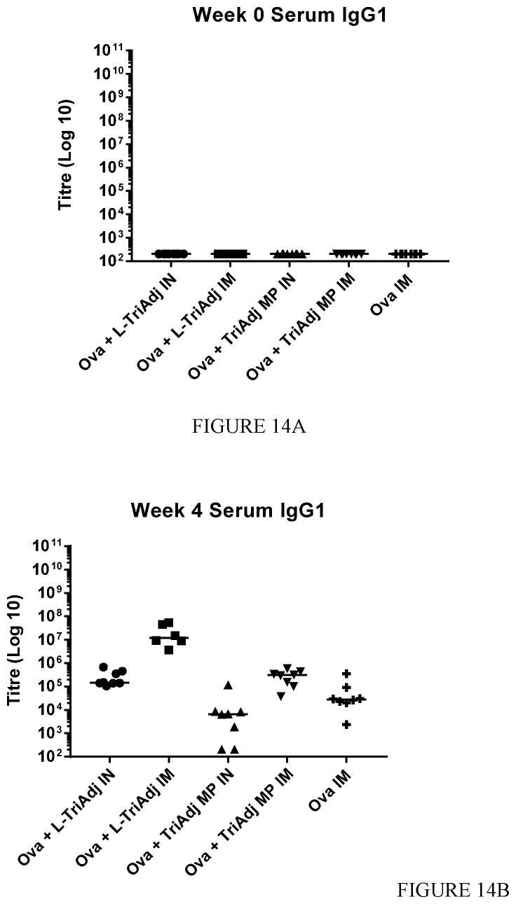

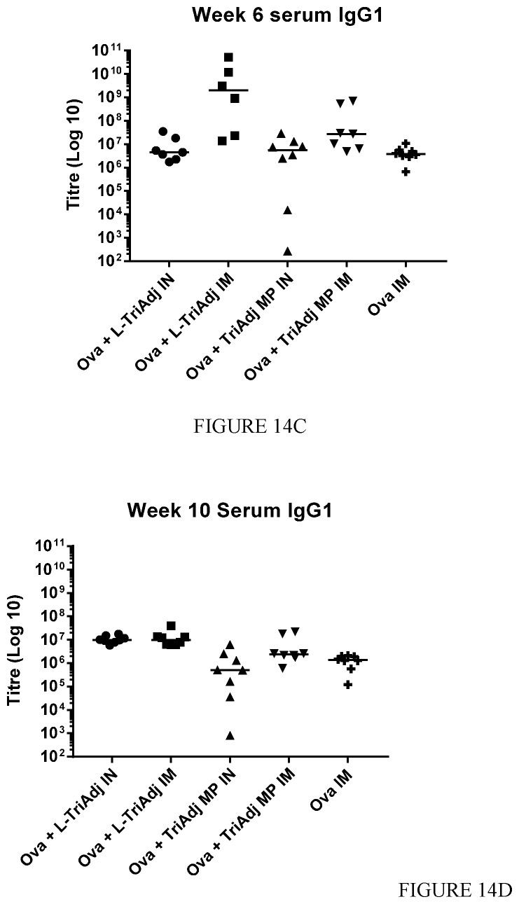

FIGS. 14-18 show the results of the third in vivo study in mice, comparing the effect of the intranasal and intramuscular routes on the immune response induced by L-TriAdj (reported as DDAB/DOPE 50:50 above) or TriAdj formulated as calcium microparticles. Ova without adjuvant served as control. The Ova antigen dose was 10 μg for all adjuvant and control groups. A booster dose was administered intranasally at week 4. Euthanasia was conducted at 10 weeks.

FIG. 14 represents the serum IgG1 levels at 0 (FIG. 14A), 4 (FIG. 14B), 6 (FIG. 14C), and 10 (FIG. 14D) weeks from mice receiving intranasal or intramuscular Ova vaccines adjuvanted with TriAdj microparticles (labelled as TriAdj) or L-TriAdj DDAB/DOPE 50:50 (labeled as L-TriAdj), as measured by ELISA.

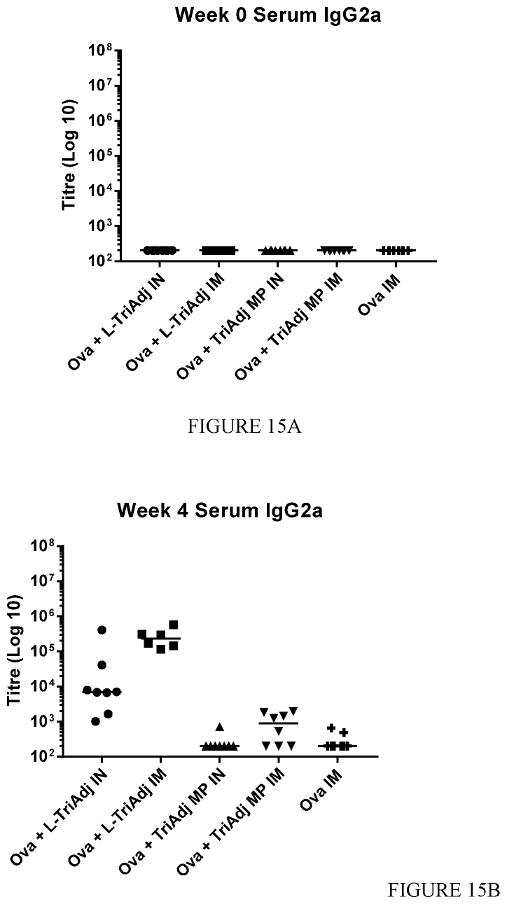

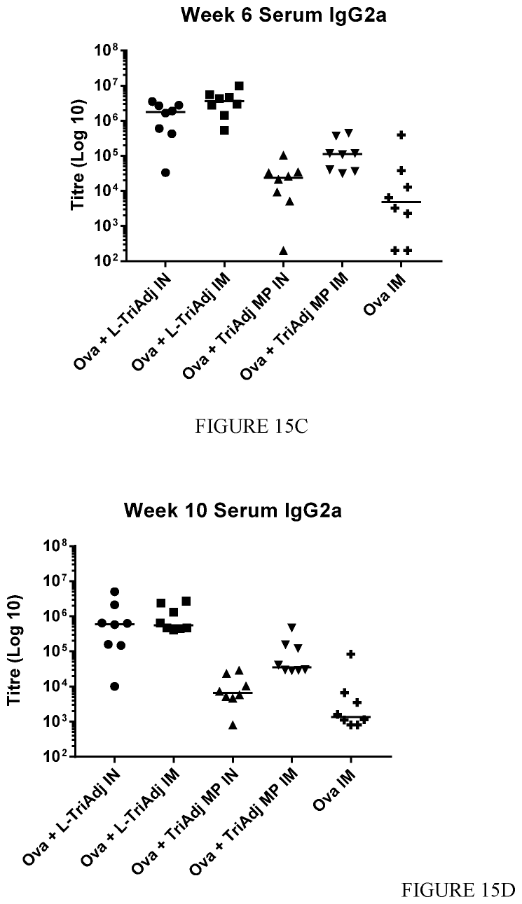

FIG. 15 represents the corresponding IgG2a serum levels from the same animals as in FIG. 14 at the same time points (FIGS. 15A-D for 0, 4, 6 and 10 weeks respectively), as measured by ELISA.

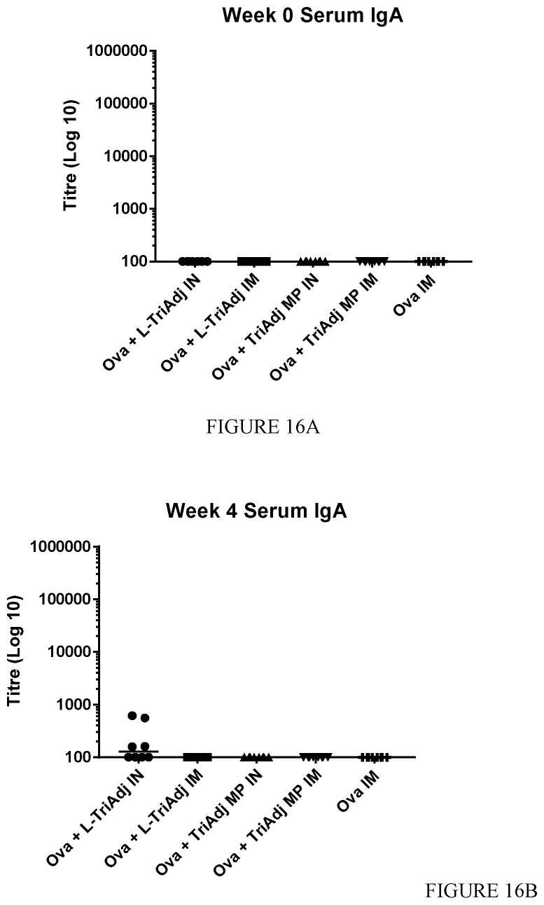

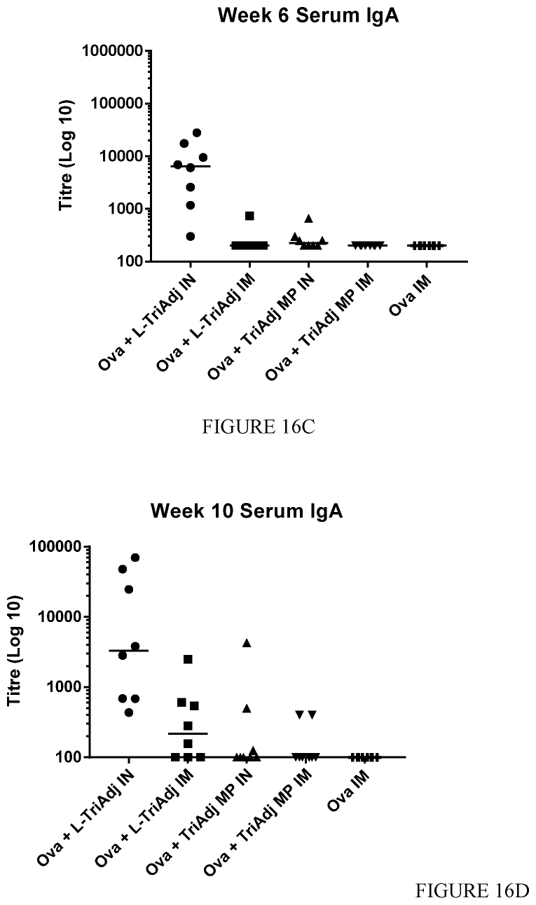

FIG. 16 represents the corresponding IgA serum levels from the same animals as in FIG. 14 at the same time points (FIGS. 16A-D for 0, 4, 6 and 10 weeks respectively), as measured by ELISA.

The IgG1 and IgG2a titres were elevated four weeks after a first immunization with 10 μg Ova formulated with the lipidic triple adjuvant delivered intramuscularly, relative to all other formulations and routes (FIGS. 14B and 15B). Two weeks after the second immunization, i.e. at 6 weeks, L-TriAdj formulation delivered intramuscularly still induced higher titres of IgG1 (FIG. 14C), and higher titres of Ig2a (FIG. 15C), than the other formulations and routes. At 6 weeks, Ig2a titres after IM immunisation of L-TriAdj formulations were higher then after IN and IM immunisation of TriAdj microparticle formulations (p<0.0001 for Ova+L-TriAdj IM vs. TriAdj IM or IN and Ova alone).

Serum IgA titres were not detected at 4 weeks after the first immunization, except in the group vaccinated intranasally with L-TriAdj (FIG. 16B). At 6 weeks, i.e. at 2 weeks after the second immunization, serum IgA titres were further elevated in the group vaccinated intranasally with L-TriAdj and were significantly higher (p<0.0001) than in any other groups (FIG. 16C). At 10 weeks, the L-TriAdj formulation delivered intranasally outperformed again all groups, including the intramuscularly delivered L-TriAdj formulation (FIG. 16D, p<0.01).

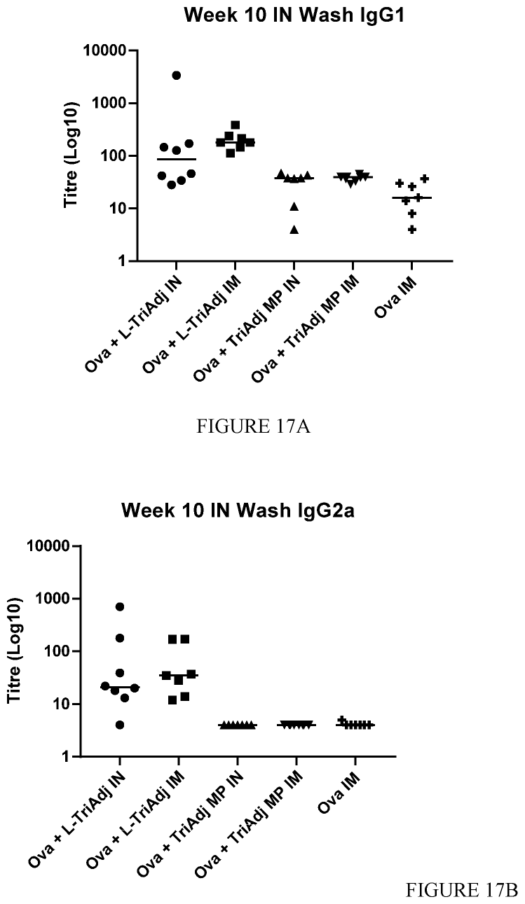

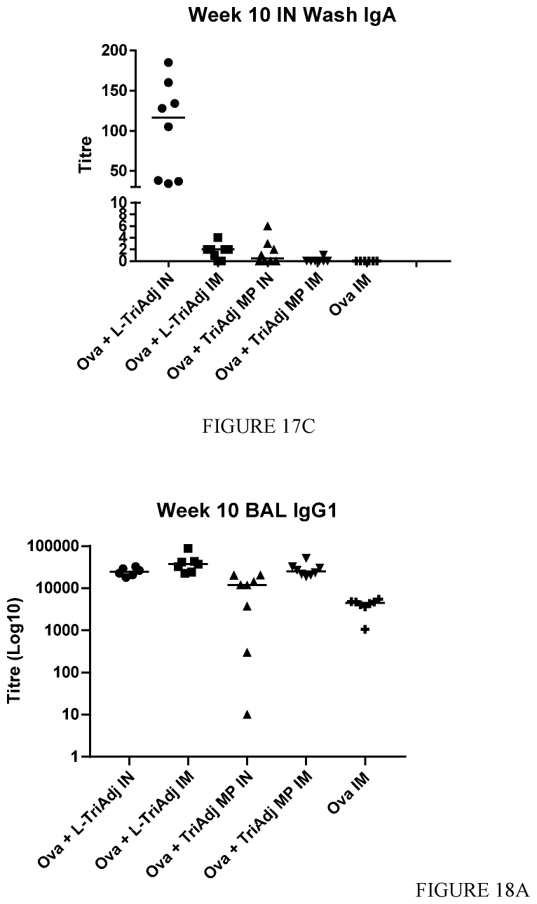

FIG. 17 represents the antibody titres detected in intranasal (IN) wash samples collected at the time of euthanasia (Week 10) from mice administered intranasal or intramuscular Ova vaccines adjuvanted with TriAdj microparticles (labelled as TriAdj) or DDAB/DOPE 50:50 (labeled as L-TriAdj), as measured by ELISA. IN wash titres of IgG1 (FIG. 17A) were found to be slightly elevated after immunization with all formulations containing TriAdj relative to immunization with Ova alone, with the highest titres being observed after IM immunization with L-TriAdj formulation. IN wash titres of IgG2a (FIG. 17B) were only detected after immunization with L-TriAdj formulations, intranasally or intramuscularly delivered. IN wash titres of IgA (FIG. 17C) were found to be elevated after immunization with the intranasally delivered L-TriAdj formulation (p<0.0001 vs. all other conditions).

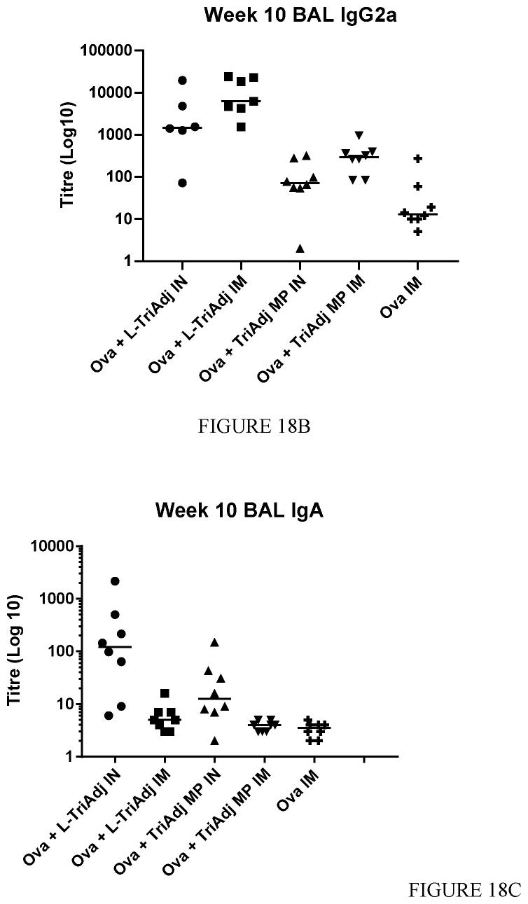

FIG. 18 represents the antibody titres detected in bronchioalveolar lavage (BAL) samples collected at the time of euthanasia (Week 10) from mice administered intranasal or intramuscular Ova vaccines adjuvanted with TriAdj microparticles (labelled as TriAdj) or DDAB/DOPE 50:50 (labeled as L-TriAdj), as measured by ELISA. BAL titres of IgG1 (FIG. 18A) were found to be elevated after immunization with all formulations containing TriAdj relative to immunization with Ova alone. BAL titres of IgG2a (FIG. 18B) showed higher levels after immunization with L-TriAdj formulations, intranasally or intramuscularly, relative to TriAdj microparticle formulations. BAL titres of IgA (FIG. 18C) were found to be elevated after immunization with the intranasally delivered formulations, especially with L-TriAdj.

Elevated IgA titres were detected in the serum of mice immunized intranasally with L-TriAdj formulations at all time points tested after the first immunization, as well as in the IN wash and BAL samples collected at 10 weeks, overall demonstrating a rapid and sustained induction of mucosal immunity.

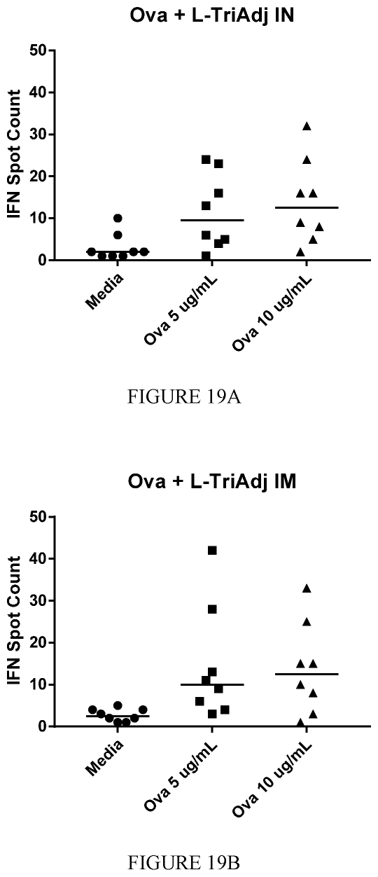

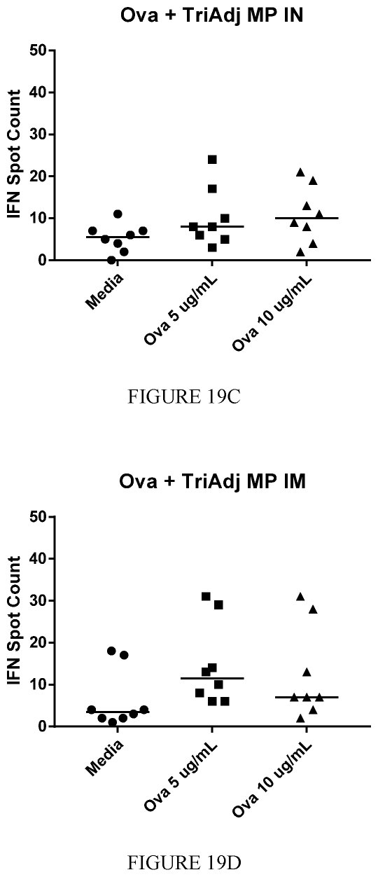

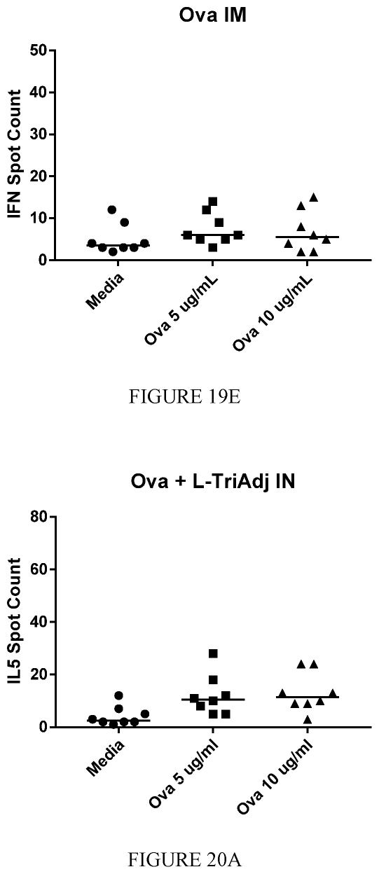

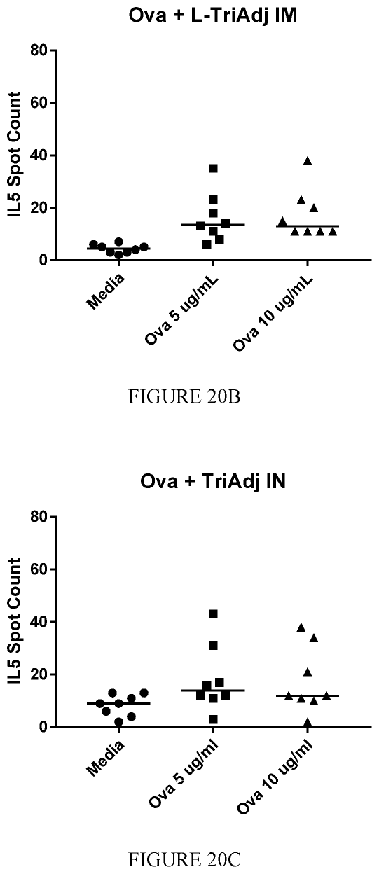

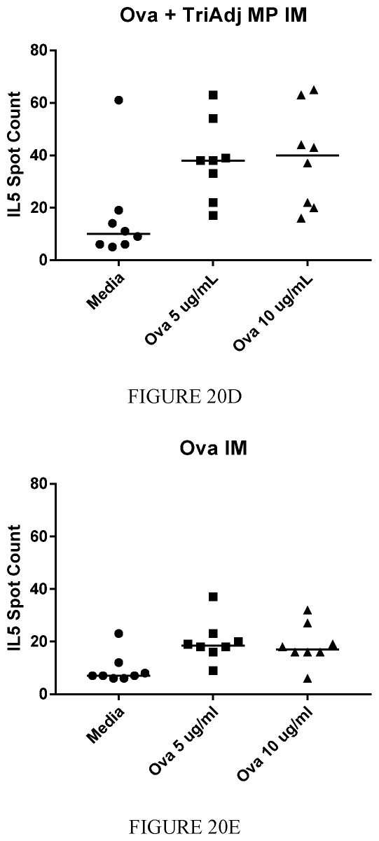

FIGS. 19 and 20 represent the ELISpot results. The spleen lymphocytes from the vaccinated mice were exposed in triplicate to 5 or 10 μg/mL ovalbumin ex vivo and secretion of IFN-γ (FIG. 19) and IL5 (FIG. 20) were measured. The ratio of these values reflects the balance of cellular (Th1) vs. humoral (Th2) type response. ELISpot results for secretion of IFN-γ from 10 μg/mL Ova-stimulated splenocytes (FIG. 19) showed more responders in groups vaccinated with adjuvanted formulations than in the group vaccinated intramuscularly with ovalbumin only (FIG. 19E), and a greater proportion of strong responders in the groups vaccinated with L-TriAdj with the intranasal (FIG. 19A) or intramuscular route (FIG. 19C), confirming the ability of L-TriAdj to induce a Th1 response. ELISpot results for secretion of IL-5 from 10 μg/mL Ova-stimulated splenocytes (FIG. 20) showed similar responders across all groups, except for the group vaccinated intramuscularly with 10 μg Ova formulated in TriAdj microparticles that showed higher response (FIG. 20D).

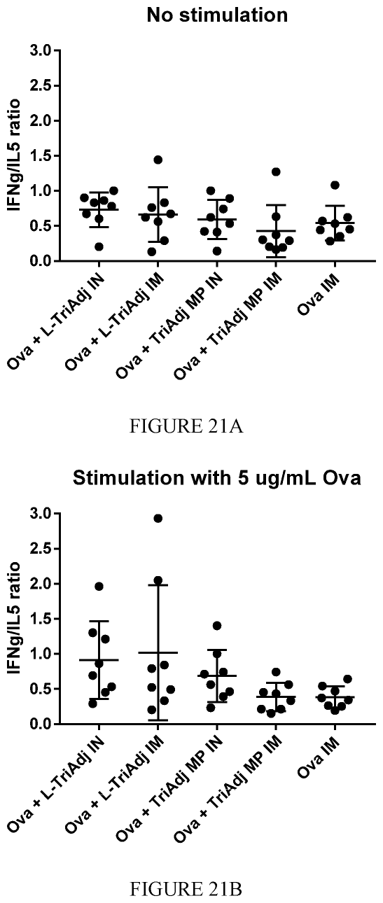

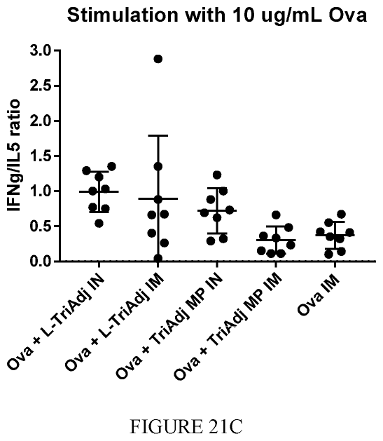

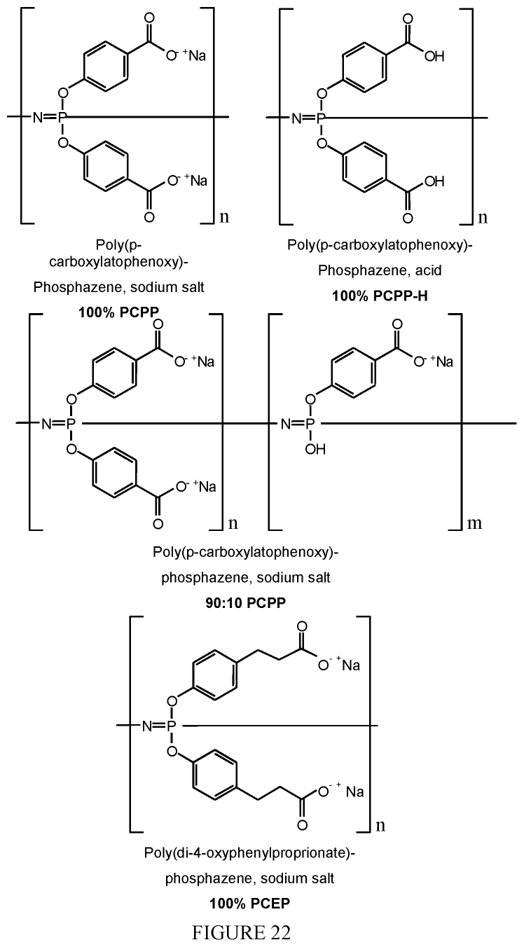

FIG. 21 represents the balance of cellular vs. humoral response as represented by IFN-γ/IL5 ratios. Stimulation with 10 μg/mL ovalbumin (FIG. 21C) induced the secretion of more IFN-γ relative to IL5 in splenocytes of mice that had been vaccinated with L-TriAdj (intranasally or intramuscularly) than in splenocytes of mice vaccinated with TriAdj microparticles or no adjuvant. These results confirmed the ability of L-TriAdj to induce a more balanced Th1/Th2 response.

In sum, the combination of lipid nanocarrier with the triple adjuvant composition undergoes a super-molecular self-assembly process which results in lipidic nanoparticles of ideal diameter and charge. The composition facilitates adherence to mucin and may permit its penetration. The lipid composition was comprised of a cationic lipid, such as DDAD, for immunostimulation and mucin binding, as well as helper lipid, such as DOPE, to aid endosomal escape. Modulation of both liposomal surface charge density and, theoretically, liposomal membrane fluidity, was achieved by inclusion of phosphatidylcholine (EPC). The assembly process of cationic liposomes and the triple adjuvant composition was reproducible and generated stable, condensed L-TriAdj particles with adjuvant activity in excess of that achieved by the triple adjuvant composition alone.

The balance of charged polyelectrolyte components incorporated into the lipidic adjuvant promoted self-assembly and condensation, and an overall cationic charge inhibited gross aggregation and facilitated mucin interaction as indicated by zeta potential alteration. The condensation of components also generated relatively small particles (<200 nm) that would be of a diameter amenable to cellular uptake. Whole-vaccine (antigen+adjuvant) size analysis and 24-hour stability indicated submicron particles as well. Ideally, the antigen and adjuvant are taken up by the same APC, so binding of the antigen to the lipidic adjuvant is advantageous.

Mixed adjuvants provide a distinct advantage by activating different aspects of the immune response and lowering the antigen dose or number of doses required to generate a response of sufficient strength to protect the host following challenge with the infectious agent. Poly(I:C) is a synthetic version of double-stranded RNA which alerts the immune system by nature of its pathogen-associated molecular pattern (PAMP), activating an immune response via Toll-like receptor 3 (TLR3). It not only drives a cytotoxic/Th1 response and production of proinflammatory cytokines, but it also modulates the duration of response, promoting apoptosis of dendritic cells (Fuertes et al., PLoS One (2011) 6:e20189), which is important for immune response resolution. PCEP is a synthetic anionic polymer with immunostimulatory properties that also serves as a polyelectrolyte binding agent (Garlapati et al., Vaccine (2011) 29:6540-6548; Mutwiri et al., Vaccine (2007) 25:1204). Another critical component of the triple adjuvant composition is the cationic innate defense regulatory (IDR) peptide 1002, which has multiple immune modulatory roles including recruitment and selective activation of neutrophils and dendritic cells (Garlapati et al., Vaccine (2011) 29:6540-6548; Nijnik et al., J. Immunol. (2010) 184:2539-2550; Garg et al., J. Gen. Virol. (2014) 95:301-306; Hancock et al., Nat. Rev. Immunol. (2016) 16:321-334). Through the use of rational proportions of cationic and helper lipid which enable mucoadhesive particle formation, established adjuvants can be enhanced by the nasal route of administration resulting in a balanced Th1/Th2 immune response in vivo. Particulate formulations also may have a depot effect, residing in the nasal tissues for an extended time for ongoing exposure.

This universal nasal adjuvant platform can be used for a wide range of vaccines which generate both local and systemic immunity, by advantageously producing mucosal immunity, which is the key to complete protection against respiratory infections.

Thus, novel adjuvant compositions and methods for treating and preventing infectious diseases are disclosed. Although preferred embodiments of the subject invention have been described in some detail, it is understood that obvious variations can be made without departing from the spirit and the scope of the invention as defined by the claims.

1. A mucoadhesive lipidic carrier system comprising: a triple adjuvant composition that comprises a host defense peptide, an immunostimulatory sequence and a polyanionic polymer, formulated with a mucoadhesive lipidic carrier, wherein said mucoadhesive lipidic carrier system is capable of enhancing an immune response to a selected antigen.

2. The mucoadhesive lipidic carrier system of claim 1, wherein said mucoadhesive lipidic carrier system is capable of enhancing the immune response to the selected antigen when administered mucosally.

3. The mucoadhesive lipidic carrier system of claim 1, wherein said mucoadhesive lipidic carrier system is capable of enhancing the immune response to the selected antigen when administered intramuscularly.

4. The mucoadhesive lipidic carrier system of claim 1, wherein the mucoadhesive lipidic carrier of the system comprises a cationic liposome.

5. The mucoadhesive lipidic carrier system of claim 1, wherein the mucoadhesive lipid carrier comprises one or more cationic lipids selected from 1,2-dioleoyl-3-trimethylammonium-propane (DOTAP); 3β-[N—(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); and ceramide carbamoyl-spermine (CCS).

6. The mucoadhesive lipidic carrier system of claim 5, wherein the lipidic carrier comprises DDAB and DOPE; DDAB, EPC and DOPE; SA and Chol; EPC and Chol; or SA/EPC and Chol.

7. The mucoadhesive lipidic carrier system of claim 1, wherein the host defense peptide is IDR-1002 (SEQ ID NO:19).

8. The mucoadhesive lipidic carrier system of claim 1, wherein the immunostimulatory sequence is polyinosinic-polycytidylic acid (poly(I:C)) or CpG.

9. The mucoadhesive lipidic carrier system of claim 1, wherein the polyanionic polymer is a polyphosphazene.

10. The mucoadhesive lipidic carrier system of claim 9, wherein the polyphosphazene is a poly(di-4-oxyphenylproprionate)phosphazene (PCEP).

11. The mucoadhesive lipidic carrier system of claim 1, wherein the antigen is from a pathogen that invades mucosal tissue.

12. The mucoadhesive lipidic carrier system of claim 11, wherein the antigen is from a virus, bacterium, parasite or fungus.

13. The mucoadhesive lipidic carrier system of claim 1, wherein said carrier system further comprises said antigen.

14. A cationic mucoadhesive liposome carrier system, wherein the system comprises (a) DDAB and DOPE; DDAB, EPC and DOPE; SA and Chol; EPC and Chol; or SA/EPC and Chol; (b) IDR-1002 (SEQ ID NO:19); (c) poly(I:C); (d) poly(di-4-oxyphenylproprionate)phosphazene (PCEP); and (e) an antigen from a pathogen that invades mucosal tissue.

15. The cationic mucoadhesive liposome carrier system of claim 14, wherein the antigen is from a virus, bacterium, parasite or fungus.

16. A composition comprising a mucoadhesive lipidic carrier system according claim 1 and a pharmaceutically acceptable excipient.

17. The composition of claim 16, wherein the average diameter of the mucoadhesive lipidic carrier systems in the composition is less than 200 nanometers.

18. A method of enhancing an immune response to a selected antigen, said method comprising administering to a subject the composition of claim 16; and a selected antigen.

19. The method of claim 18, wherein the administering is done mucosally.

20. The method of claim 18, wherein the administering is done intranasally.

21. The method of claim 18, wherein the administering is done intramuscularly.
